SEQUENCE LISTING (20 sequences)

SEQ ID NO: 1 - DNA, 24 bp, synthetic construct
  source 1..24: mol_type = other DNA
actggtggagtatttgatagtgta

SEQ ID NO: 2 - DNA, 20 bp, synthetic construct
  source 1..20: mol_type = other DNA
gtcctgcaccagtaatatgc

SEQ ID NO: 3 - DNA, 17 bp, synthetic construct
  source 1..17: mol_type = other DNA
aggcctgctgaaaatga

SEQ ID NO: 4 - DNA, 18 bp, synthetic construct
  source 1..18: mol_type = other DNA
aggcctgctgaaaatgac

SEQ ID NO: 5 - DNA, 20 bp, synthetic construct
  source 1..20: mol_type = other DNA
ttggatcatattcgtccaca

SEQ ID NO: 6 - DNA, 22 bp, synthetic construct
  source 1..22: mol_type = other DNA
tgttggatcatattcgtccaca

SEQ ID NO: 7 - DNA, 20 bp, synthetic construct
  source 1..20: mol_type = other DNA
aaacttgtggtagttggagc

SEQ ID NO: 8 - DNA, 49 bp, synthetic construct
  source 1..49: mol_type = other DNA
  modified_base 31: mod_base = OTHER; note = Fluorescein amidite thymine
  modified_base 32: mod_base = OTHER; note = Tetrahydrofuran guanine
  modified_base 33: mod_base = OTHER; note = Black Hole Quencher thymine
gactgaatataaacttgtggtagttggagctgtggcgtaggcaagagtg

SEQ ID NO: 9 - DNA, 100 bp, synthetic construct
  source 1..100: mol_type = other DNA
tataaggcctgctgaaaatgactgaatataaacttgtggtagttggagctggtggcgtaggcaagagtgccttgacgatacagctaattcagaatcattt

SEQ ID NO: 10 - DNA, 100 bp, synthetic construct
  source 1..100: mol_type = other DNA
aaatgattctgaattagctgtatcgtcaaggcactcttgcctacgccaccagctccaactaccacaagtttatattcagtcattttcagcaggccttata

SEQ ID NO: 11 - DNA, 100 bp, synthetic construct
  source 1..100: mol_type = other DNA
tataaggcctgctgaaaatgactgaatataaacttgtggtagttggagctgttggcgtaggcaagagtgccttgacgatacagctaattcagaatcattt

SEQ ID NO: 12 - DNA, 100 bp, synthetic construct
  source 1..100: mol_type = other DNA
aaatgattctgaattagctgtatcgtcaaggcactcttgcctacgccaacagctccaactaccacaagtttatattcagtcattttcagcaggccttata

SEQ ID NO: 13 - DNA, 100 bp, synthetic construct
  source 1..100: mol_type = other DNA
tataaggcctgctgaaaatgactgaatataaacttgtggtagttggagctgatggcgtaggcaagagtgccttgacgatacagctaattcagaatcattt

SEQ ID NO: 14 - DNA, 100 bp, synthetic construct
  source 1..100: mol_type = other DNA
aaatgattctgaattagctgtatcgtcaaggcactcttgcctacgccatcagctccaactaccacaagtttatattcagtcattttcagcaggccttata

SEQ ID NO: 15 - DNA, 17 bp, synthetic construct
  source 1..17: mol_type = other DNA
ttggatcatattcgtcc

SEQ ID NO: 16 - DNA, 307 bp, Homo sapiens
  source 1..307: mol_type = genomic DNA
tttttcatataaaggtgagtttgtattaaaaggtactggtggagtatttgatagtgtattaaccttatgtgtgacatgttctaatatagtcacattttcattatttttattataaggcctgctgaaaatgactgaatataaacttgtggtagttggagctggtggcgtaggcaagagtgccttgacgatacagctaattcagaatcattttgtggacgaatatgatccaacaatagaggtaaatcttgttttaatatgcatattactggtgcaggaccattctttgatacagataaaggtttctctg

SEQ ID NO: 17 - DNA, 28 bp, Homo sapiens
  source 1..28: mol_type = genomic DNA
aaacttgtggtagttggagctggtggcg

SEQ ID NO: 18 - DNA, 28 bp, Homo sapiens
  source 1..28: mol_type = genomic DNA
aaacttgtggtagttggagctgttggcg

SEQ ID NO: 19 - DNA, 177 bp, Homo sapiens
  source 1..177: mol_type = genomic DNA
agtcacattttcattatttttattataaggcctgctgaaaatgactgaatataaacttgtggtagttggagctggtggcgtaggcaagagtgccttgacgatacagctaattcagaatcattttgtggacgaatatgatccaacaatagaggtaaatcttgttttaatatgcatatt

SEQ ID NO: 20 - RNA, 20 nt, synthetic construct
  source 1..20: mol_type = other RNA
aaacttgtggtagttggagc

(19) United States
(12) Patent Application Publication
(10) Pub. No.: US 2023/0052289 A1
(43) Pub. Date: Feb. 16, 2023

(54) PROGRAMMABLE ENZYME-ASSISTED SELECTIVE EXPONENTIAL AMPLIFICATION FOR SENSITIVE DETECTION OF RARE MUTANT ALLELES

(71) Applicant: The Trustees of the University of Pennsylvania, Philadelphia, PA (US)

(72) Inventors: Haim H. Bau, Swarthmore, PA (US); Jinzhao Song, Philadelphia, PA (US); Junman Chen, Philadelphia, PA (US)

(21) Appl. No.: 17/871,702

(22) Filed: Jul. 22, 2022

Related U.S. Application Data
(60) Provisional application No. 63/225,370, filed on Jul. 23, 2021.
(60) Provisional application No. 63/236,410, filed on Aug. 24, 2021.

(51) Int. Cl. C12N 15/90; C12N 15/10; G01N 33/542; C12N 9/22

(52) U.S. Cl. CPC C12N 15/902; C12N 15/102; G01N 33/542; C12N 9/22; C12N 2310/20

(57) ABSTRACT

Described is an assay termed Programmable Enzyme-Assisted Selective Exponential Amplification (PASEA) that concurrently amplifies both wild type and mutant alleles while selectively cleaving the former. With time, the rare mutant alleles dominate, and are readily detectable by direct detection, Sanger sequencing, and other readily available methods. Also described are point-of-care assays and microfluidic devices for performing PASEA.

### CROSS-REFERENCE TO RELATED APPLICATIONS

The present application claims priority to and the benefit of U.S. patent application No. 63/225,370, “CRISPR Cas9—Mediated Selective Isothermal Amplification For Sensitive Detection Of Rare Mutant Alleles” (filed Jul. 23, 2021) and U.S. patent application No. 63/236,410, “Programmable Enzyme-Assisted Selective Exponential Amplification for Sensitive Detection of Rare Mutant Alleles” (filed Aug. 24, 2021), the entireties of which applications are incorporated herein by reference for any and all purposes.

### GOVERNMENT RIGHTS

This invention was made with government support under CA228614 and TW011190 awarded by the National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Oct. 13, 2022, is named 103241_006857_SL.xml and is 21,311 bytes in size.

### TECHNICAL FIELD

This invention is generally related to the field of detecting low frequency nucleic acids in a sample

### BACKGROUND

Somatic mutations strongly correlate with tumorigenesis and are central for pathological diagnosis and personalized therapies. The detection and genetic profiling of somatic mutant alleles (MA) in “liquid biopsy” is challenged by their low abundance and sequence homology with the vast background of wild type (WT) nucleic acids from healthy cells, especially at early stages of disease and during the evolution of drug resistant mutations. Recently, programmable endonucleases such as CRISPR-Cas (Gu et al., Genome Biol 2016; 17:41) and prokaryotic Argonaute (Song et al., Nucleic Acids Res 2020; 48:e19) have been successfully used to remove background nucleic acids and enrich rare mutant allele fractions (MAFs), enabling their detection with deep next generation sequencing (NOS). However, these assays are limited by the absence of restriction endonuclease recognition sites (Song et al., Nucleic Acids Res 2016; 44:e146), futile binding events (Sternberg et al., Nature 2014; 507:62-7), and off-target cleavage (Sternberg et al., Nature 2014; 507:62-7). Although rounds of selective depletion of WT followed with PCR overcame some of these shortcomings (Lee et al., Oncogene 2017; 36:6823-9), high sensitivity detection of mutant alleles still requires deep NGS ((Lee et al., Oncogene 2017; 36:6823-9), rendering these methods laborious, time-consuming, and expensive.

### SUMMARY

Disclosed is a method of selective amplification of a target nucleic acid in a sample, comprising:

amplifying the target nucleic acid in an amplification reaction comprising:
a) the target nucleic acid;
b) a non-target nucleic acid comprising protospacer adjacent motif (PAM);
b) a guide nucleic acid comprising protospacer target sequence that forms a guide/non-target hybrid with the non-target nucleic acid;
d) an endonuclease having an affinity for the guide/non-target hybrid; and
c) a polymerase;
   wherein amplifying is amplifying with the polymerase for up to about 120 min, and
   wherein the target nucleic acid in the sample is present at a frequency of about 0.001% of nucleic acids in the sample or greater.

Also disclosed is a method of detecting a target nucleic acid in a sample, comprising:

amplifying the target nucleic acid in an amplification reaction comprising:
a) the target nucleic acid;
b) a non-target nucleic acid comprising protospacer adjacent motif (PAM);
c) a guide nucleic acid comprising protospacer target sequence that forms a guide/non-target hybrid with the non-target nucleic acid;
d) an endonuclease having an affinity for the guide/non-target hybrid;
e) a nucleic acid probe substantially specific to the target nucleic acid; and
f) a polymerase.

Further disclosed is a point-of-care assay comprising real-time detection of target nucleic acids in a sample.

It is understood that the methods described herein can process either a single target nucleic acid or a panel of target nucleic acids in the same amplification reaction.

### BRIEF DESCRIPTION OF THE DRAWINGS

FIG. 1A-1C: Programmable Enzyme-Assisted Selective Exponential Amplification (PASEA) enriches exponentially mutant alleles' fraction. Directed by a single-guide RNA (sgRNA), Cas9 selectively cleaves WT alleles with protospacer adjacent motif (PAM) site while sparing oncogenic mutation lacking PAM. While both WT and mutant alleles amplify, the rate of amplification of mutant alleles far exceeds that of the WT. Nearly all the amplification products are mutant alleles.

FIG. 2A, FIG. 2Bi, and FIG. 2Bi: PASEA exponentially enriches mutant allele fraction (MAF, also referred to herein as mutant allele frequency). (FIG. 2A) A mixture of WT and KRAS G12V (MAF 5%) was subjected to PASEA incubation for various time spans. The number of amplicons is inferred from the threshold time of pre-calibrated qPCR (FIG. 8). N=3. (FIG. 2Bi) Sanger sequencing of PASEA products. (FIG. 2Bii) Estimated MAF as a function of PASEA incubation time. Ribonucleoprotein (RNP) concentration is 0.1 μM.

FIG. 3A, FIG. 3B, FIG. 3Bi, and FIG. 3Bii: PASEA exhibits high sensitivity. (FIG. 3A) Electropherogram of PASEA products when MAF=0%, 0.1%, 0.1%, 1%, and 5%, (FIGS. 3B1-3Biii) Sanger sequencing of PASEA products: (FIG. 3Bi) Summary of Sanger sequencing results. (FIG. 3Bi) Average MAF following PASEA incubation. (FIG. 3Biii) Sanger sequencing data. The total gDNA in each sample is 60 ng. The PASEA incubation time is 20 minutes with 0.1 μM RNP.

FIG. 4A, FIG. 4B, and FIG. 4C: Recombinase polymerase amplification (RPA) probe enables real-time detection. (FIG. 4A) Schematics of the Exo-RPA probe. (FIG. 4B) KRAS gene (SEQ ID NO: 16) with the locations of the primers, sgRNA protospacer ( ), and Exo-RPA probe indicated. (FIG. 4C) Real-time RPA monitoring of serially diluted, wild-type alleles in the absence of RNP. Probe concentration: 240 nM. FIG. 4D is a diagram showing the principle of real-time PASEA. Directed by a single-stranded guided RNA (sgRNA), Cas9 selectively cleaves WT alleles with protospacer adjacent motif (PAM) site while sparing oncogenic mutation lacking PAM (FIG. 4E, SEQ ID NOs: 17, 18 and 20). The dotted frame illustrates WT and mutant allele sequences, the location of PAM site in the WT KRAS and its absence in KRAS G12. Cleavage takes place between the third nucleotide and the fourth nucleotide upstream from the PAM site. While PASEA amplifies both WT and mutant alleles, the rate of amplification of mutant alleles far exceeds that of the WT, resulting in a product dominated by mutant alleles. Exo-probe indicates the number of amplicons and enables quantification in real time (FIG. 4F).

FIG. 5A, FIG. 5B, FIG. 5C, FIG. 5D, FIG. 5E, FIG. 5F, FIG. 5G, and FIG. 5H: Real-time PASEA. Amplification curves of cell-free DNA (cfDNA) (FIG. 5A). mRNA (FIG. 5B), mixture of cfDNA and mRNA (FIG. 5C) with various MAF. Threshold time as a function of initial MAF when detecting cfDNA (FIG. 5D); mRNA (FIG. 5E); and a mixture of cfDNA and mRNA (FIG. 5F). Fluorescence intensity at 45 min as a function of MAF when detecting cfDNA (FIG. 5G) and mRNA (FIG. 51).

FIG. 6A, FIG. 6B, FIG. 6C, FIG. 6D, FIG. 6E, and FIG. 6F: Real-time PASEA of gDNA and cfDNA in clinical samples. (FIG. 6A and FIG. 6C) F₄₅ values of patient samples (83) compared with tissue NGS genotyping (“gold standard”). (FIG. 61) Real-time PASEA amplification curves of 83 samples in (FIG. 6A and FIG. 6C). (FIG. 6D) Workflow of blood testing with PASEA. (FIG. 6E) NGS KRAS positive blood sample. (FIG. 6F) Real-time PASEA amplification curves of cfDNA samples.

FIG. 7A, FIG. 7B, and FIG. 7C: RPA buffer is compatible with Cas9 cleavage. (FIG. 7A) WT and mutant allele sequences (SEQ ID NOs: 17 and 18) showing the presence of PAM site in the WT KR4S and its absence in KRAS G12 (sgRNA is SEQ ID NO: 20). Cleavage takes place between the third nucleotide and the fourth nucleotide upstream from the PAM site. (FIG. 7B) Polyacrylamide gel electropherograms of 1 hour cleavage products (copies/μL) of 250 nM, 100 bp synthetic WT and mutant allele suspended in RPA rehydration buffer without primers: in the absence of RNP (−); in the presence 2.5 μM RNP (+); and in the presence of 2.5 μM RNP (+) and RPA reaction mix. Relatively high concentrations of DNA (and thus of RNP) are used to produce visible gel bands. (FIG. 7C) Cleavage efficiency of WT KRAS and G12V dsDNA incubated with the assay in (FIG. 78). The band intensity is normalized with the corresponding value in the absence of cleavage. Gel images were analyzed with BioRad Gel Doc XR+ imaging system.

FIG. 8: qPCR calibration curve for KRAS alleles. The threshold cycle Cq as a function of logarithmic copy number of templates (N=3).

FIG. 9A, FIG. 9B, FIG. 9C, FIG. 9D, and FIG. 9E: Enrichment efficiency as a function of RNP concentration. Sanger sequencing data. 10 min PASEA incubation time. 60 ng genomic DNA containing 5% of KRAS G12V.

FIG. 10A, FIG. JOB, FIG. 10C, FIG. 10D, FIG. 10E, FIG. 10F, FIG. 10G, FIG. 10H, and FIG. 10I: PASEA's performance as a function of mutant allele fraction (MAF) with incubation time for 10, 20, and 30 minutes. Sanger sequencing data. The total gDNA in each sample is 60 ng.

FIG. 11A, FIG. 11B FIG. 11C, FIG. 11D, FIG. 11E, and FIG. 11F: RPA probe design and evaluation for real-time detection. (FIG. 11A), (FIG. 11C) and (FIG. 11E) Real-time RPA monitoring of serially diluted wild-type alleles in the absence of RNP with the designed Exo-RPA probes. (FIG. 11B), (FIG. 11D) and (FIG. 11F) KRAS gene (SEQ ID NO: 16) with the different locations of the designed Exo-RPA probe and locations of the primers and sgRNA protospacer. The probe in (FIG. 11F) (Table 1) was selected for real-time PASEA. Probe concentration: 240 nM.

FIG. 12A, FIG. 12B, and FIG. 12C: Optimization of probe concentration for real-time PASEA. Real-time PASEA with various Exo-probe concentrations: 600 nM (FIG. 12A), 240 nM (FIG. 12B), 120 nM (FIG. 12C). KRAS samples containing 5% and 0% G12V alleles were used as templates. 0.1 μM RNP.

FIG. 13A, FIG. 13B, FIG. 13C, FIG. 13D, FIG. 13E, and FIG. 13F: Optimization of RNP concentration for real-time PASEA. Amplification curves of gDNA samples containing 5% and 0% KRAS G12V with 0.1 μM (FIG. 13A), 0.08 μM (FIG. 13B). 0.05 μM (FIG. 13C), and 0 μM (FIG. 13D) RNP. Amplification curves of cfDNA samples containing 0/n, 0.1%, 1%, and 5% KRAS G12V with 0.1 μM (FIG. 13E) and 0.08 μM RNP (FIG. 13F). NTC is non-template control. 240 μM Exo-probe.

FIG. 14A, FIG. 14B, FIG. 14C, FIG. 14D, FIG. 14E, and FIG. 14F: Selection of RPA primers for both ctDNA and ctRNA. (FIG. 14A, SEQ ID NO: 19) KRAS gene sequence around exon 2, showing the locations of sgRNA, protospacer, Exo-RPA probe, and the primers. The homologous sequence (125 bp) with cDNA is shown in black letters, which contains exon 2 (italic, 122 bp). (FIG. 14B)-(FIG. 14E) Real-time RPA curves of serially diluted, wild-type alleles in the absence of RNP with primer sets (Table 1) RPA-ct-Fw-1/RPA-ct-Rv-1 (FIG. 14B), RPA-ct-Fw-1/RPA-ct-Rv-2 (FIG. 14C), RPA-ct-Fw-2/RPA-ct-Rv-2 (FIG. 14D), and RPA-ct-Fw-2/RPA-ct-Rv-1 (FIG. 14E). Threshold time as a function of total nucleic acid concentration in the sample (primer set RPA-ct-Fw-1/RPA-ct-Rv-2) (FIG. 14F). Probe concentration: 240 nM. The threshold time is defined as the time until the normalized fluorescent signal exceeds ˜10% of its saturation emission intensity.

FIG. 15A and FIG. 15B: The emission intensity (F₄₅) correlates well with threshold time. (a) ctDNA detection. (b) mRNA detection. The data of the emission intensity and the threshold time corresponds to FIG. 5D, FIG. 5E, FIG. 5G, and FIG. 5H.

FIG. 16A and FIG. 16B: Real-time PASEA of gDNA and cIDNA in clinical samples. (FIG. 16A) F₄₅ values of PASEA tests (97) compared with tissue ARMS-PCR genotyping (“gold standard”). Dashed horizontal line and the symbol “x” denote, respectively, F₄₅ cutoff value and median F₄₅ value (Table 2). (FIG. 16B) Real-time PASEA compared with tissue ARMS-PCR genotyping.

FIG. 17: The emission intensity (F₄₅) correlates well with threshold time for clinical sample detection. The data of the emission intensity and the threshold time are from FIG. 6B.

FIG. 18: Custom-made, multifunctional microfluidic (MIAR) chip (inset) (Song et al., Analytical chemistry 2018; 90 7:4823-31) (4) and portable heating and imaging platform for isothermal amplification and fluorescence detection.

FIG. 19A and FIG. 19B On Chip, Real-time PASEA. (FIG. 19A): Real time amplification curve of standard cfDNA with various MAF as detected with portable USB camera (FIG. 18). (FIG. 19B) Fluorescence emission images detected with a camera from the microfluidic chip (FIG. 18).

FIG. 20A, FIG. 20B, FIG. 20C, FIG. 20D, FIG. 20E, and FIG. 20F: Sanger sequencing sonograms of samples incubated with CRISPR Cas9 (DASH) for 20 min in the absence of RPA amplification. DASH has successfully increased mutant allele fraction by a factor of 10 for the sample with MAF 1%.

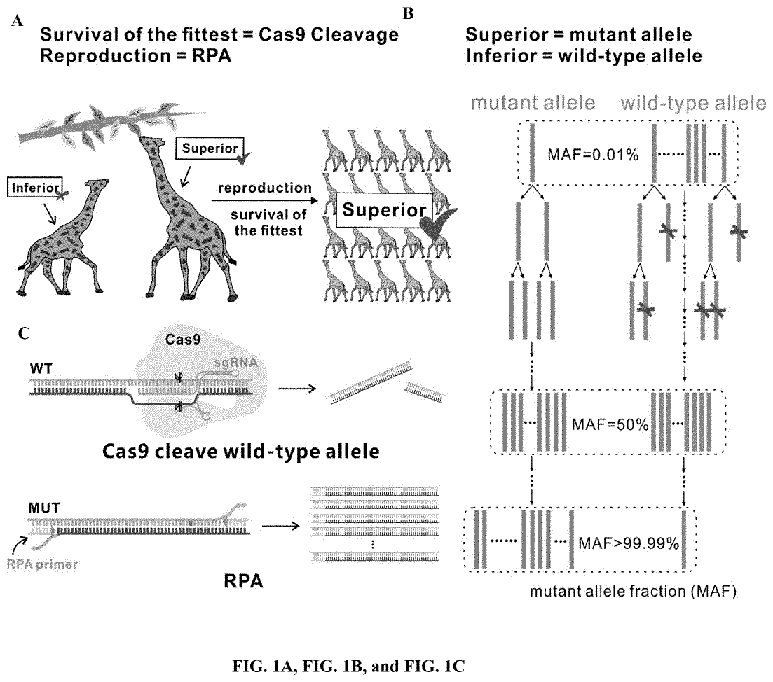

### DETAILED DESCRIPTION

Described is an assay termed Programmable Enzyme-Assisted Selective Exponential Amplification (PASEA) that concurrently amplifies both wild type and mutant alleles while selectively cleaving the former. With time, the rare mutant alleles dominate, and are readily detectable by direct detection, Sanger sequencing, and other readily available methods. Also described are point-of-care assays and microfluidic devices for performing PASEA.

The disclosed methods of selective amplification of a target nucleic acid in a sample comprise:

amplifying the target nucleic acid in an amplification reaction comprising:
a) the target nucleic acid;
b) a non-target nucleic acid comprising protospacer adjacent motif (PAM);
b) a guide nucleic acid comprising protospacer target sequence that forms a guide/non-target hybrid with the non-target nucleic acid;
d) an endonuclease having an affinity for the guide/non-target hybrid; and
e) a polymerase;
   wherein amplifying is amplifying with the polymerase for up to about 60 min, or up to about 120 min, and
   wherein the target nucleic acid in the sample is present at a frequency of about 0.001% of nucleic acids in the sample or greater.

The guide nucleic acid can be incubated with the endonuclease prior to amplifying.

The target nucleic acid can be at a frequency between about 0.001% and about 15% of combined target and non-target nucleic acids in the sample, at a frequency between about 0.001% and 10% of combined target and non-target nucleic acids in the sample, at a frequency between about 0.01% and 5% of combined target and non-target nucleic acids in the sample, or at a frequency between about 0.01% and about 1% of combined target and non-target nucleic acids in the sample.

In some embodiments, the target nucleic acid does not comprise PAM.

In some embodiments, the method can increase the frequency of the target nucleic acid between 10 and 10,000 fold.

In some embodiments, the method can increase the frequency of the target nucleic acid between 100 and 10,000 fold.

In some embodiments, the method can increase the frequency of the target nucleic acid between 1000 and 10,000 fold.

The method step of amplifying the target nucleic acid can comprise amplifying with polymerase chain reaction (PCR), loop-mediated isothermal amplification (LAMP), recombinase polymerase amplification (RPA), any other polymerase amplification methods known in the art, or any combination thereof.

The target nucleic acid can comprise a mutation associated with a disease or can be a variant associated with a disease. Suitable diseases include cancers and genetic diseases.

In some embodiments, the target nucleic acid and the non-target nucleic acid are from different strains of a pathogen.

In some embodiments, the target nucleic acid induces drug-resistance.

The endonuclease of the methods can be a Cas endonuclease. Suitable Cas endonucleases for use in the methods include Cas12a (from bacterial species Francisella novicida, Acidaminococcus or Lachnospiraceae), SaCas9 (from bacterial species Staphylococcus aureus), CjCas9 (from bacterial species Campylobacter jejuni), SpCas9 (from bacterial species Streptococcus pyogenes), and NmCas9 (from bacterial species Neisseria meningitidis).

The guide nucleic acid can be RNA. The target nucleic acid can be a DNA or an RNA.

In some embodiments, amplifying is by using a polymerase having an optimal operating temperature substantially similar to an optimal operating temperature of the endonuclease. For example, a polymerase having an optimal operating temperature substantially similar to an optimal operating temperature of the endonuclease can include a polymerase having between about 2° C. and about 20° C. difference, between about 2° C. and about 15° C. difference, between about 2° C. and about 10° C. difference, or between about 2° C. and about 5° C. difference from the optimal operating temperature of the endonuclease.

The amplification can produce amplification reaction products as a library of amplicons for sequencing. The amplification reaction products can be detected using a method selected from the group consisting of probe hybridization, chain termination sequencing, next generation sequencing, restriction enzyme digestion, and electrophoresis.

The methods can have between about 80% and 100% sensitivity of detecting target nucleic acids having a frequency of between about 0.1% and 5% in the sample.

Also disclosed are methods of detecting a target nucleic acid in a sample, comprising:

amplifying the target nucleic acid in an amplification reaction comprising:
a) the target nucleic acid;
b) a non-target nucleic acid comprising protospacer adjacent motif (PAM);
c) a guide nucleic acid comprising protospacer target sequence that forms a guide/non-target hybrid with the non-target nucleic acid;
d) an endonuclease having an affinity for the guide/non-target hybrid;
e) a nucleic acid probe substantially specific to the target nucleic acid; and
   f) a polymerase.

The nucleic acid probe can comprise a 3′ blocker, a detectable label comprising a fluorophore, a quencher, and an abasic nucleotide analogue between the fluorophore and the quencher. The step of amplifying can be by isothermal amplification for up to about 120 min and wherein the target nucleic acid in the sample is at a frequency of about 0.001% or greater. For example, the step of amplifying can be by isothermal amplification for up to about 3 min, up to about 5 min, up to about 8 min, up to about 10 min. up to about 15 min, up to about 20 min, up to about 25, min, up to about 30 min, up to about 35, min, up to about 40 min, up to about 45, min, up to about 50 min, up to about 55, min, up to about 60 min, up to about 65, min, up to about 70 min. up to about 75, min, up to about 80 min, up to about 85, min, up to about 90 min, up to about 95, min. up to about 100 min, up to about 105, min, up to about 110 min, up to about 115, min, or up to about 120 min, and wherein the target nucleic acid in the sample is at a frequency of about 0.001% or greater. The step of amplifying can be by isothermal amplification for a time period between about 3 min and about 120 min, such as between about 3 min and about 100 min, between about 3 min and about 80 min, between about 3 min and about 60 min, between about 3 min and about 50 min, between about 3 min and about 40 min, between about 3 min and about 30 min, between about 3 min and about 20 min, such as between about 5 min and about 20 min, between about 5 min and about 15 min, and wherein the target nucleic acid in the sample is at a frequency of about 0.001% or greater.

The probe can comprise a nucleic acid sequence substantially specific to nucleic acid sequence of the target nucleic acid and/or of the non-target nucleic acid. The fluorophore and the quencher of the probe can be separated by a length of between 2 and 10 nucleic acids. The step of the amplifying in the methods produces amplicons of the target nucleic acid. The fluorophore can be released from the probe, thereby reporting on the presence of an amplicon having nucleic acid sequence substantially similar to nucleic acid sequence of the target nucleic acid and/or of the non-target nucleic acid.

In some embodiments, the detecting step of the method can comprise detecting fluorophore.

In some embodiments, the abasic nucleotide analogue comprises tetrahydrofuran residue. THF.

In some embodiments, the fluorophore emits light at a wavelength between about 480 nm and 700 nm.

in some embodiments, the quencher comprises absorbs light at a wavelength between about 480 nm and 700 nm. The methods of detecting a target nucleic acid can further comprise an exonuclease, and, optionally, wherein the exonuclease is an exonuclease III enzyme. The target nucleic acid can be at a frequency between about 0.001% and about 5% of the nucleic acids in the sample. In some embodiments, the target nucleic acid does not comprise PAM. In some embodiments, the frequency of the target nucleic acid is increased between 500 and 10,000 fold.

The methods of detecting can comprise amplifying the target nucleic acid comprises polymerase chain reaction (PCR), loop-mediated isothermal amplification (LAMP), recombinase polymerase amplification (RPA), or any combination thereof.

The methods of detecting can detect the target nucleic acid comprising a mutation associated with a disease or can be a variant associated with a disease. Suitable diseases include cancers and genetic diseases.

In some embodiments of the method of detecting, the target nucleic acid and the non-target nucleic acid are from different strains of a pathogen. In some embodiments of the methods of detecting, the target nucleic acid induces drug-resistance.

The endonuclease in the methods of detecting can be a Cas endonuclease. Suitable Cas endonucleases for use in the methods of detecting include Cas12a (from bacterial species Francisella novicida, Acidaminococcus or Lachnospiraceae), SaCas9 (from bacterial species Staphylococcus aureus), CjCas9 (from bacterial species Campylobacter jejuni), SpCas9 (from bacterial species Streptococcus pyogenes), and NmCas9 (from bacterial species Neisseria meningitidis).

In some embodiments of the methods of detecting, the guide nucleic acid is RNA. In some embodiments of the methods of detecting, the guide nucleic acid is incubated with the endonuclease prior to amplifying. In some embodiments of the methods of detecting, the target nucleic acid is a DNA or an RNA.

In some embodiments of the methods of detecting, amplifying is by using a polymerase having an optimal operating temperature substantially similar to an optimal operating temperature of the endonuclease. For example, a polymerase having an optimal operating temperature substantially similar to an optimal operating temperature of the endonuclease can include a polymerase having between about 2° C. and about 20° C. difference, between about 2° C. and about 15° C. difference, between about 2° C. and about 10° C. difference, or between about 2° C. and about 5° C. difference from the optimal operating temperature of the endonuclease.

The methods of detecting can provide amplification reaction products as a library of amplicons for sequencing. In some embodiments of the methods of detecting, amplification reaction products are detected using a method selected from the group consisting of probe hybridization, chain termination sequencing, next generation sequencing, restriction enzyme digestion, and electrophoresis. In some embodiments, the method has between about 80% and 100% sensitivity of real-time detecting of low frequency target nucleic acids having frequency of between about 0.1% and 5%.

Also provided are methods of multiplexed selective amplification of two or more different target nucleic acids in the same amplification reaction. Also provided are methods of multiplexed detection of two or more different target nucleic acids in the same amplification reaction. The two or more target nucleic acids can comprise different mutant alleles. In some embodiments, the frequencies of the two or more different target nucleic acids are increased concurrently. In some embodiments, the two or more different target nucleic acids comprise biomarkers for specific types of cancer.

Also provided is a point-of-care assays comprising real-time detection of target nucleic acids in a sample according to any one of the disclosed methods. The point-of-care assays can be performed in a microfluidic device.

PASEA can increase the MAF of mutants associated with various diseases and disorders. The examples show PASEA increases the MAF of KRAS G12 mutations, including G12C, G12D, G12V, G12S, and G12R mutations (Table 2). This illustrative data based on KRAS G12 is illustrative only and is not limiting, as PASEA can increase the MAF of one or more mutant genes from a panel of mutations and biomarkers associated with a disease. Different disease-causing mutations are known, including mutations of genes ALK, BRAF, EGFR, ERBB2, HRAS, KRAS, MET, and NRAS in lung cancer, mutation of genes BRCA1, BRCA2, CHEK2, ATM PALB2, BARL1, RAD51D and MSH6 in breast cancer, and mutations of APC, ATM, AXIN2, BLM, BMPR1A, BUB1B, CDH1, CEP57, CHEK2, ENG, EPCAM, FLCN, GALNT12, GREM1, MLH1, MLH3, MSH2, MSH3, MSH6, MUTYH, NTHL1, PMS2, POLD1, POLE, PTEN, RNF43, RPS20, SMAD4, STK11, and TP53 in colon cancer (Kanta et al., Annals of Oncology, 28, supplement 7, 15, 2017; Lee at al., Oncogene, 36, 6823-6829 (2017); Chen et al., Molecular Oncology, 13, 1490-1502 (2019); Mayo Clinic Laboratories, Targeted Gene Regions Interrogated by Lung Panel; Cooch et al., JAMA Oncology, 9, 1190-1196 (2017)). PASEA can be applied to the foregoing panels as well as other panels.

### Illustrative Disclosure

Described is a new assay dubbed Programmable Enzyme-Assisted Selective Exponential Amplification (PASEA) that concurrently amplifies both wild type and mutant alleles while selectively cleaving the former. With time, the rare mutant alleles dominate, and are readily detectable.

As the Examples show, the CRISPR-Cas9 endonuclease programmed to cleave wild type alleles is combined with isothermal Recombinase Polymerase Amplification (RPA). Cas9 programmed with sgRNA selectively cleaves wild type allele that contains NGG PAM site while sparing oncogenic mutant alleles such as KRAS G12V lacking PAM. Thus, PASEA preferentially amplifies mutant alleles, increasing the fraction of ultrarare somatic mutant alleles within a short time to levels detectable with inexpensive sequencers such as Sanger in a single step.

CRISPR-Cas9 is compatible with RPA. In the absence of primers, ribonucleopratein (RNP, Cas9 protein combined with the targeting sgRNA) in RPA reaction buffer cleaves nearly 70% of the 100 bp WT synthetic dsDNA KRAS (250 nM) that contains the PAM site within one-hour of incubation.

PASEA provides unprecedented MAF enrichment. MAF of 5% is barely visible in the Sanger sensorgram in the absence of PASEA; appears clearly after 3 min of PASEA; and dominates the signal after 5 min or longer of PASEA. When MAF=1%, incubation time of 10 min suffices to deplete the WT to undetectable level in the Sanger sensorgram. When MAF=0.1%, both the WT and MA signatures are evident in the sensorgram after 10 min of PASEA but only the MA is evident after 20 min of PASEA.

To assess PASEA's sensitivity, a standard genomic DNA panel with 0%, 0.01%. 0.1%, 1%, and 5% KRAS G12V MAFs was subjected to 20-min PASEA incubation. Sanger sequencing of PASEA products identified the presence of KRAS G12V in all samples with MAF=0.1%. 1%, and 5% (N=3) and in 70% of the samples with MAF=0.01%, (N=10), without any false positives. PASEA increased the MAFs to nearly 100% in the 5% (20-fold enrichment) and 1% (100-fold enrichment) samples; to 80% (800-fold enrichment) in the 0.1% samples; and to 40%(4000-fold enrichment) in the 0.01% samples. The samples included 60 ng of DNA (˜17,400 copies). MAF of 0.01% is equivalent to ˜2 copies of mutant allele per sample. This suggests that PASEA enabled enrichment and detection of as few as 2 copies of mutant alleles and that the 70% sensitivity observed when MAF=0.01% results from sampling error, as a few of the samples may have not contained any mutant alleles, and not from any PASEA deficiency.

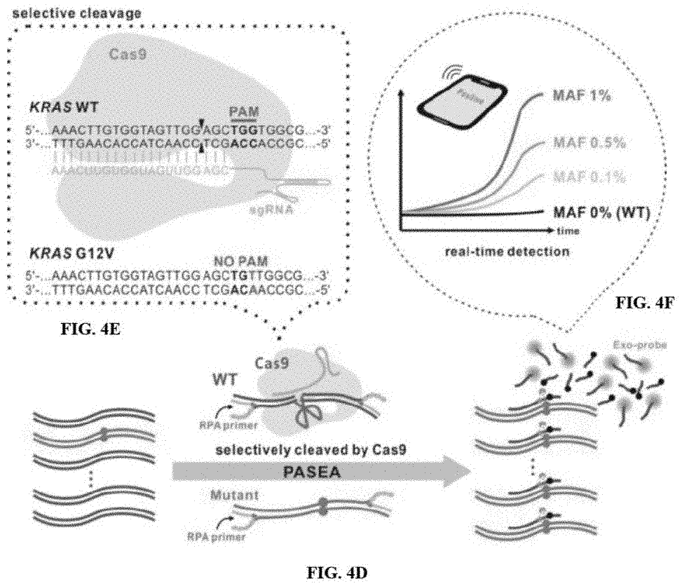

PASEA's very high enrichment capability enables preparing libraries for rapid, low-cost sequencers such as Sanger. With Exo-RPA probes, PASEA identifies the presence of mutant alleles in real time and can be employed at the point-of-care (POC) (FIGS. 4D-4F). Furthermore, PASEA may be able to accommodate other programmable endonucleases such as Argonautes (Song et al., Nucleic Acids Res 2020; 48:e19) that do not require the presence of PAM and are therefore more versatile.

### Aspects

Aspect 1. A method of selective amplification of a target nucleic acid in a sample, comprising:

amplifying the target nucleic acid in an amplification reaction comprising:
a) the target nucleic acid;
b) a non-target nucleic acid comprising protospacer adjacent motif (PAM);
b) a guide nucleic acid comprising protospacer target sequence that forms a guide/non-target hybrid with the non-target nucleic acid;
d) an endonuclease having an affinity for the guide/non-target hybrid: and
e) a polymerase;
   wherein amplifying is amplifying with the polymerase for up to about 120 min, and
   wherein the target nucleic acid in the sample is present at a frequency of about 0.001% of nucleic acids in the sample or greater.

Aspect 2. The method of aspect 1, wherein the guide nucleic acid is incubated with the endonuclease prior to amplifying.

Aspect 3. The method of aspect 1 or aspect 2, wherein the target nucleic acid is at a frequency between about 0.001% and about 15% of combined target and non-target nucleic acids in the sample.

Aspect 4 The method of any one of aspects 1-3, wherein the target nucleic acid is at a frequency between about 0.01% and 5% of combined target and non-target nucleic acids in the sample.

Aspect 5. The method of any one of aspects 1-4, wherein the target nucleic acid is at a frequency between about 0.01% and about 1% of combined target and non-target nucleic acids in the sample.

Aspect 6. The method of any one of aspects 1-5, wherein the target nucleic acid does not comprise PAM.

Aspect 7. The method of any one of aspects 1-6, wherein the frequency of the target nucleic acid is increased between 10 and 10.000 fold.

Aspect 8. The method of any one of aspects 1-7, wherein the frequency of the target nucleic acid is increased between 100 and 10,000 fold.

Aspect 9. The method of any one of aspects 1-8, wherein the frequency of the target nucleic acid is increased between 1000 and 10,000 fold.

Aspect 10. The method of any one of aspects 1-9, wherein amplifying the target nucleic acid comprises polymerase chain reaction (PCR), loop-mediated isothermal amplification (LAMP), recombinase polymerase amplification (RPA), or any combination thereof.

Aspect 11. The method of any one of aspects 1-10, wherein the target nucleic acid comprises a mutation or is a variant associated with a disease.

Aspect 12. The method of any one of aspects 1-11, wherein the target nucleic acid and the non-target nucleic acid are from different strains of a pathogen.

Aspect 13. The method of any one of aspects 1-11, wherein the target nucleic acid induces drug-resistance.

Aspect 14. The method of any one of aspects 1-13, wherein the endonuclease is a Cas endonuclease.

Aspect 15. The method of aspect 14, wherein the Cas endonuclease is selected from the group consisting of Cas12a (from bacterial species Francisella novicida, Acidaminococcus or Lachnospiraceae), SaCas9 (from bacterial species Staphylococcus aureus), CjCas9 (from bacterial species Campylobacter jejuni), SpCas9 (from bacterial species Streptococcus pyogenes), and NmCas9 (from bacterial species Neisseria meningitidis).

Aspect 16. The method of any one of aspects 1-15, wherein the guide nucleic acid is RNA.

Aspect 17. The method of anyone of aspects 1-16, wherein the target nucleic acid is a DNA or an RNA.

Aspect 18. The method of any one of aspects 1-17, wherein amplifying is by using a polymerase having an optimal operating temperature substantially similar to an optimal operating temperature of the endonuclease.

Aspect 19. The method of any of the aspects 1-18, wherein amplification reaction products provide a library of amplicons for sequencing.

Aspect 20. The method of any of the aspects 1-19, wherein amplification reaction products are detected using a method selected from the group consisting of probe hybridization, chain termination sequencing, next generation sequencing, restriction enzyme digestion, and electrophoresis.

Aspect 21. The method of any one of aspects 1-20, wherein the method has between about 80% and 100% sensitivity of detecting target nucleic acids having a frequency of between about 0.1% and 5% in the sample.

Aspect 22. A method of detecting a target nucleic acid in a sample, comprising:

amplifying the target nucleic acid in an amplification reaction comprising:
a) the target nucleic acid;
b) a non-target nucleic acid comprising protospacer adjacent motif (PAM);
c) a guide nucleic acid comprising protospacer target sequence that forms a guide/non-target hybrid with the non-target nucleic acid;
d) an endonuclease having an affinity for the guide/non-target hybrid;
e) a nucleic acid probe substantially specific to the target nucleic acid; and
f) a polymerase.

Aspect 23. The method of aspect 22, wherein the nucleic acid probe comprises a 3′ blocker, a detectable label comprising a fluorophore, a quencher, and an abasic nucleotide analogue between the fluorophore and the quencher.

Aspect 24. The method of aspect 22 or 23, wherein amplifying is by isothermal amplification for up to about 120 min, and wherein the target nucleic acid in the sample is at a frequency of about 0.001% or greater.

Aspect 25. The method of any one of aspects 22-24, wherein the probe comprises a nucleic acid sequence substantially specific to nucleic acid sequence of the target nucleic acid and/or of the non-target nucleic acid.

Aspect 26. The method of any one of aspects 23-25, wherein the fluorophore and the quencher are separated by a length of between 2 and 10 nucleic acids.

Aspect 27. The method of any one of aspects 22-26, wherein the amplifying produces amplicons of the target nucleic acid.

Aspect 28. The method of any one of aspects 23-27, wherein the fluorophore is released from the probe, thereby reporting on the presence of an amplicon having nucleic acid sequence substantially similar to nucleic acid sequence of the target nucleic acid and/or of the non-target nucleic acid.

Aspect 29. The method of any one of aspects 22-28, wherein detecting comprises detecting fluorophore emission from the probe.

Aspect 30. The method of any one of aspects 23-29, wherein the abasic nucleotide analogue comprises tetrahydrofuran residue, THF.

Aspect 31. The method of any one of aspects 23-30, wherein the fluorophore emits light at a wavelength between about 480 nm and 700 nm.

Aspect 32. The method of any one of aspects 23-31, wherein the quencher comprises absorbs light at a wavelength between about 480 nm and 700 nm.

Aspect 33. The method of any one of aspects 22-32, further comprising an exonuclease, and, optionally, wherein the exonuclease is an exonuclease III enzyme.

Aspect 34. The method of any one of aspects 22-33, wherein the target nucleic acid is at a frequency between about 0.001% and about 15% of the nucleic acids in the sample.

Aspect 35. The method of any one of aspects 22-34, wherein the target nucleic acid does not comprise PAM.

Aspect 36. The method of any one of aspects 22-35, wherein frequency of the target nucleic acid is increased between 500 and 10,000 fold.

Aspect 37. The method of any one of aspects 22-36, wherein amplifying the target nucleic acid comprises polymerase chain reaction (PCR), loop-mediated isothermal amplification (LAMP), recombinase polymerase amplification (RPA), or any combination thereof.

Aspect 38. The method of any one of aspects 22-37, wherein the target nucleic acid comprises a mutation or is a variant associated with a disease.

Aspect 39. The method of any one of aspects 22-38, wherein the target nucleic acid and the non-target nucleic acid are from different strains of a pathogen.

Aspect 40. The method of any one of aspects 22-39, wherein the target nucleic acid induces drug-resistance.

Aspect 41. The method of any one of aspects 22-40, wherein the endonuclease is a Cas endonuclease.

Aspect 42. The method of aspect 41, wherein the Cas endonuclease is selected from the group consisting of Cas12a (from bacterial species Francisella novicida, Acidaminococcus or Lachnospiraceae), SaCas9 (from bacterial species Staphylococcus aureus), CjCas9 (from bacterial species Campylobacter jejuni). SpCas9 (from bacterial species Streptococcus pyogenes), and NmCas9 (from bacterial species Neisseria meningitidis).

Aspect 43. The method of any one of aspects 22-42, wherein the guide nucleic acid is RNA.

Aspect 44. The method of any one of aspects 22-43, wherein the guide nucleic acid is incubated with the endonuclease prior to amplifying.

Aspect 45. The method of any one of aspects 22-44, wherein the target nucleic acid is a DNA or an RNA.

Aspect 46. The method of any one of aspects 22-45, wherein amplifying is by using a polymerase having an optimal operating temperature substantially similar to an optimal operating temperature of the endonuclease.

Aspect 47. The method of any one of aspects 22-46, wherein amplification reaction products provide a library of amplicons for sequencing.

Aspect 48. The method of any one of aspects 22-47, wherein amplification reaction products are detected using a method selected from the group consisting of probe hybridization, chain termination sequencing, next generation sequencing, restriction enzyme digestion, and electrophoresis.

Aspect 49. The method of any one of aspects 22-48, wherein the method has between about 80% and 100% sensitivity of real-time detecting of low frequency target nucleic acids having frequency of between about 0.1% and 5%.

Aspect 50. The method of any one of aspects 1-49, wherein the method comprises amplifying two or more target nucleic acids in the same amplification reaction.

Aspect 51. The method of aspect 50, wherein the two or more target nucleic acids comprise mutant alleles.

Aspect 52. The method of aspect 50 or 51, wherein the frequencies of the two or more target nucleic acids are increased concurrently.

Aspect 53. The method of any one of aspects 50-52, wherein the two or more target nucleic acids comprise a biomarker for a specific type of cancer.

Aspect 54. A point-of-care assay comprising real-time detection of target nucleic acids in a sample according to the method of any one of aspects 22-53.

Aspect 55. The point-of-care assay of aspect 54, performed in a microfluidic device.

### EXAMPLES

Somatic mutations strongly correlate with tumorigenesis and play a key role in pathological diagnosis and in guiding therapies. The detection and genetic profiling of rare somatic mutant alleles (MA) in cancer biopsy and “liquid biopsy” is challenged by their low abundance and sequence homology with the vast background of wild type (WVT) nucleic acids from healthy cells, especially during early stages of disease and during the evolution of drug resistant mutations. To detect somatic mutations, it is necessary to improve the signal to noise ratio. Several methods have been developed to enrich the mutant allele fraction (MAF) by either suppressing the amplification of WT alleles during or cleaving WT alleles before PCR. The first class of methods includes amplification refractory mutation system (ARMS) (1), blocker PCR (2), clamping PCR (3), co-amplification at lower denaturation temperature-PCR (COLD-PCR) (4), and super-selective primer PCR (5). The second, more recent, class of methods utilize programmable enzymes such as clustered regularly interspaced short palindromic repeats-associated proteins (CRISPR-Cas)-based Depletion of Abundant Sequences by Hybridization (DASH) (6), and prokaryotic Argonaute (pAgo)-based NAVIGATER (7, 8).

CRISPR-Cas and pAgo play a key role in the defense mechanisms of various bacteria and archaea (9-12) and have recently been leveraged in molecular biotechnology. Guided by synthetic oligonucleotides, these endonucleases target and cleave complementary sequences with high specificity (13-17). These programmable endonucleases remove the dominant and interfering (background) wild-type sequences to facilitate detection of scarce mutant alleles. While greatly improving the sensitivity of downstream genotyping methods, the efficacy of existing endonuclease-based enrichment assays is compromised by a significant fraction of unfertile binding events between enzyme and target, which due to the slow dissociation rate of Cas9 leave targets protected from cleavage (18) and by non-specific off-target cleavage that depletes precious biomarkers (19). To partially overcome these shortcomings, researchers have employed rounds of selective depletion of WT followed by polymerase chain reaction (PCR), enabling tens-fold enrichment of the fractions of mutant alleles (19). Despite these improvements, high sensitivity of mutant allele detection still requires the use of deep NGS (19), rendering these methods laborious, time-consuming, and expensive. A single-step method that enriches the MAF to enable its detection by inexpensive and rapid means is highly desirable.

Inspired by Darwin theory of the survival of the fittest, described is a new assay dubbed Programmable Enzyme-Assisted Selective Exponential Amplification (PASEA) that concurrently amplifies both wild type and mutant alleles while selectively cleaving the former. Given time, the variant that exhibits a superior trait (the mutant allele) will dominate. PASEA exponentially increases the copy number of mutant alleles to nearly 100% of the amplicons. PASEA must be implemented with temperature-matched polymerase and endonuclease. Herein, CRISPR-Cas9 programmed to cleave wild type alleles is used in combination with isothermal recombinase polymerase amplification (RPA). With specifically designed primers, PASEA amplifies both RNA and DNA, providing an unprecedented enrichment, increasing the fraction of ultra rare somatic mutant alleles within 20 min to a level detectable with inexpensive sequencers such as Sanger in a single step. Furthermore, with the aid of a custom-designed Exo-RPA probe, PASEA identifies the presence of mutant alleles in real time, suggesting the possibility of mutant allele detection at the point-of-care (POC) in a closed tube or in a microfluidic chip. The real time PASEA performed on par with next-generation sequencing (NGS) and amplification refractory mutation system (ARMS)-PCR when testing 108 clinical tissue samples and 10 blood samples from cancer patients.

### Example 1. PASEA

Materials and Methods

Samples

Standard genomic DNA (gDNA), cell-free DNA (cfDNA), and RNA samples. Standard gDNA and cfDNA were purchased from Horizon Discovery. Total RNA was extracted with RNeasy® mini kit (Qiagen, Valencia, Calif., USA) per manufacturer's protocol from Human cancer cell lines U87-MG (WT KRAS mRNA) and ASPC1 (KRAS G12D mRNA).

Patient tissue DNA samples. Tissue samples from 62 colorectal cancer patients, 45 lung cancer patients, and 1 pancreatic cancer patient (Table 2) were collected from Cancer Hospital, Chinese Academy of Medical Sciences by either resection or biopsy under the IRB-approved protocol (20/383-2579). Genomic DNA (gDNA) was extracted with DNeasy® Blood & Tissue Kit (Qiagen, Valencia, Calif., USA). Subsequently, the extracted gDNA was quantified with NanoDrop™ spectrophotometer and diluted to 10 ng/μL.

Patient cfDNA samples. 10 lung cancer patient blood samples were obtained from Cancer Hospital. Chinese Academy of Medical Sciences under the IRB-approved protocol (20/383-2579), cfDNA was extracted with QIAamp® Circulating Nucleic Acid kit (Qiagen, Valencia, Calif., LISA). Subsequently, the extracted cfDNA was quantified with NanoDrop™ spectrophotometer and qualified with clinical NGS.

Preparation of Cas9-sgRNA Ribonucleoprotein Complex

S.p. Cas9 Nuclease V3 (Cas9) and sgRNA (protospacer sequence: 5′-AAACTTGTGGTAGTTGGAGC-3′ (SEQ ID NO: 7)) were purchased from Integrated DNA Technologies (Coralville, US). The sgRNA and Cas9 were mixed in Working buffer (30 mM HEPES pH 7.5, KCl 150 mM) in equimolar amounts and incubated at room temperature for 10 min to form the Cas9-sgRNA ribonucleoprotein (RNP) complex.

Programmable Enzyme Based Exponential Enrichment Assay

Exponential enrichment was carried out in a 10 μL rehydrated (Ix) RPA reaction mix (Twistamp® Basic kit) containing extra 0.1 μM RNP, 0.5 μM RPA primers (5′-ACTGGTGGAGTAGTTTGATAGTGTA-3′(SEQ ID NO: 1). 5′-GTCCTGCACCAGTAATATGC-3′(SEQ ID NO: 2)), 14 mM Magnesium Acetate (MgOAc), and 60 ng standard genomic DNA (Horizon Discovery) with various MAF (0%. 0.01%, 0.1, 1%, 5%). The reaction mixes were incubated at 37° C. for 3-20 minutes and then at 95° C. for 10 min to stop the reaction by denaturing enzymes. Subsequently, 1 μL RNase A (10 mg/mL) was added to digest sgRNA with incubation at room temperature for 10 min. And 1 μL Proteinase K (20 mg/mL) was added to digest the RNase A and Cas9 endonuclease with incubation at 56° C. for 30 min. Then, the Proteinase K was deactivated at 95° C. for 10 min for the following analysis.

Programmable Enzyme Based Linear Enrichment Assay

For comparison, linear enrichment assay was also carried out with Twistamp® Basic kit (TwistDx, Cambridge, UK) but without adding primers. 10 μL reaction mix contains 0.1 μM RNPs, 14 mM Magnesium Acetate (MgOAc), and 60 ng standard genomic DNA with various MAF (0%, 0.01%, 0.1, 1%, 5%) into rehydrated (1×) Twistamp® Basic RPA reaction mix, and mixed thoroughly. The reaction mixes were incubated at 37° C. for 20 minutes and then stopped. sgRNA and enzymes were removed or deactivated as described above for following analysis.

Quantitative PCR of Enrichment Product

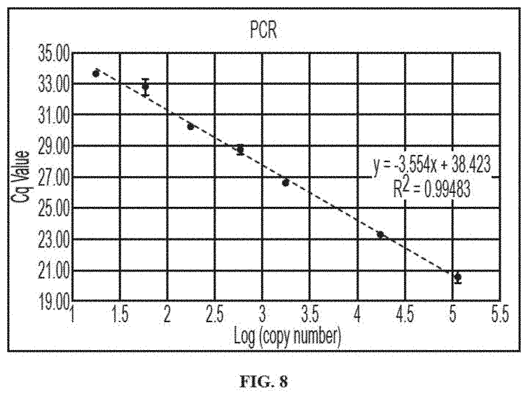

For relative quantitation of products of CRISPR-RPA, a standard calibration curve was prepared with certain concentration of targets with qPCR (DiaCarta, Inc.) according to the manufacture's instruction (DiaCarta, Inc.) (FIG. 8). Enrichment products were relatively quantified by the same qPCR protocol. 10 μL reaction mix, containing 2 μL of the 10⁴-10⁶ fold diluted, exponential enrichment products, and 1 μL. PCR primer/probe mix in 1×PCR Master Mix was prepared. Reactions (qPCR) were amplified with a BioRad Thermal Cycler (BioRad, Model CFX96) with a temperature profile of 95° C. for 5 minutes, followed by 45 cycles of amplification (95° C. for 20 seconds, 70° C. for 40 seconds, 60° C. for 30 seconds, and 72° C. for 30 seconds).

Sanger Sequencing

The qPCR products were inspected for quality and yield by running 5 μl in 2.2% agarose Lonza FlashGel DNA Cassette. After treating with Exo-CIP Rapid PCR Cleanup Kit to remove the remaining primers, probe, and dNTP, the products were processed for Sanger sequencing at the Penn Genomic Analysis Core with the reverse KRAS PCR primer 5′-IIGGATCATATTCGTCC-3((SEQ ID NO: 15)). Mutation’ Surveyor Software was used for mutation quantification.

Real-Time PASEA for Detection of Rare Mutant Alleles

First, the real-time detection assay was tested by using genomic DNA as target. The experiment was carried out in 10 μL rehydrated (Ix) RPA reaction mix (TwistAmp® Exo kit) containing 60 ng standard genomic DNA, 420 nM each of RPA primers (RPA-g-Fw and RPA-g-Rv in Table 1), 14 mM Magnesium Acetate (MgOAc), 0.1 μM RNP, 240 μM Exo-RPA Probe (Table 1). The experiment was carried out on ice. After vortexing, the reaction mix was placed into a BioRad Thermal Cycler (BioRad, Model CPX96) for isothermal amplification under 37° C. with plate-read each 30 sec. For detection of clinical tissue samples, 2 μL of extracted gDNA (10 ng/μL) was added into real-time PASEA reaction mixture.

TABLE 1
Sequences of RPA primers, RNA guide,
Exo_probe, and synthetic KRAS template.

| | | SEQ |
|---|---|---|
| Name | Sequence (from 5′ to 3′) | ID NO |
| RPA-g-Fw-1 | ACTGGTGGAGTATTTGATAGTGTA | 1 |
| RPA-g-Rv-1 | GTCCTGCACCAGTAATATGC | 2 |
| RPA-ct-Fw-1 | AGGCCTGCTGAAAATGA | 3 |
| RPA-ct-Fw-2 | AGGCCTTGAAAATGAC | 4 |
| RPA-ct-Rv-1 | TTGGATCATATTCGTCCACA | 5 |
| RPA-ct-Rv-2 | TGTTGGATCATATTCGTCCACA | 6 |
| sgRNA | AAACTTGTGGTAGTTGGAGC | 7 |
| protospacer | | |
| sequence | | |
| Exo-RPA-Probe | GACTGAATATAAACTTGTGGTAGTTGGAGC | 8 |
| | [FAM-dT]G[THF][BHQ-dT]GGCGTAGG | |
| | CAAGAGTG | |
| 100 nt WT-S | tataaggcctgctgaaaatgactgaatat | 9 |
| | aaacttgtggtagttggagctggtggcgt | |
| | aggcaagagtgccttgacgatacagctaa | |
| | ttcagaatcattt | |
| 100 nt WT-As | aaatgattctgaattagetgtategtcaa | 10 |
| | ggcactettgectacgceaceagctceaa | |
| | ctaccacaagtttatattcagtcattttc | |
| | agcaggccttata | |
| 100 nt G12V-S | tataaggcctgctgaaaatgactgaatat | 11 |
| | aaacttgtggtagttggagctgttggcgt | |
| | aggcaagagtgccttgacgatacagctaa | |
| | ttcagaatcattt | |
| 100 nt G12-AS | aaatgattctgaattagctgtatcgtcaa | 12 |
| | ggcactcttgcctacgccaacagctccaa | |
| | ctaccacaagtttatattcagtcatttca | |
| | gcaggccttata | |
| 100 nt G12D-S | tataaggcctgctgaaaatgactgaatat | 13 |
| | caaattgtggtagttggagctgatggcgt | |
| | aggcaagagtgccttgacgatacagctaa | |
| | ttcagaatcattt | |
| 100 nt G12D-AS | aaatgattctgaattagctgtatcgtcaa | 14 |
| | ggcactcttgcctacgccatcagctccaa | |
| | ctaccacaagtttatattcagtcattttc | |
| | agcaggccttata | |

RPA Primer Development for Detection of Both Cell-Free DNA and RNA

To enable real-time detection of both KRAS DNA and RNA mutant alleles, the primers were designed based on a shared sequence (125 bp) in both gDNA and cDNA in around a single KRAS exon that is harboring G12 mutations. Since the average size of cfDNA is ˜160 bp (Underhill el al., PLoS Genetics 2016; 12 7:e1006162: Sato et al., Oncotarget 2018; 9 61), the amplicon was shortened when the primers were designed. The sequences and positions of the RPA primers (RPA-ct-Fw and RPA-ct-Rv primers) were respectively listed in Table 1. The performance of these primers was tested with the standard protocol of TwistAmp® Exo kit with various amounts (4 ng, 400 pg, 40 pg, 4 pg, and 0 pg) of standard cfDNA (Horizon Discovery) as target.

The primer set with lowest limit of detection was selected for real-time detection of rare mutant alleles with the same protocol as described above, except reducing RNP concentration to 0.08 μM. Instead of 60 ng genomic DNA, 20 ng standard cfDNA (Horizon Discovery), 400 ng mRNA, or the mixture of 10 ng standard cfDNA and 200 ng mRNA with various MAF (0%, 0.01%, 0.1, 1%, 5%) were added into rehydrated (1×) Twistamp® Exo RPA buffer as target. When testing mRNA, 0.2 μL AMV Reverse Transcriptase (10 U/μL) was included. When testing clinical samples, 2 μL diluted genomic DNA (10 ng/μL) from tissue sample or 3 μL extracted cfDNA from blood sample were added into real-time PASEA reaction mixture.

Point-of-Care Detection of Rare Mutant Alleles on a Microfluidic Chip

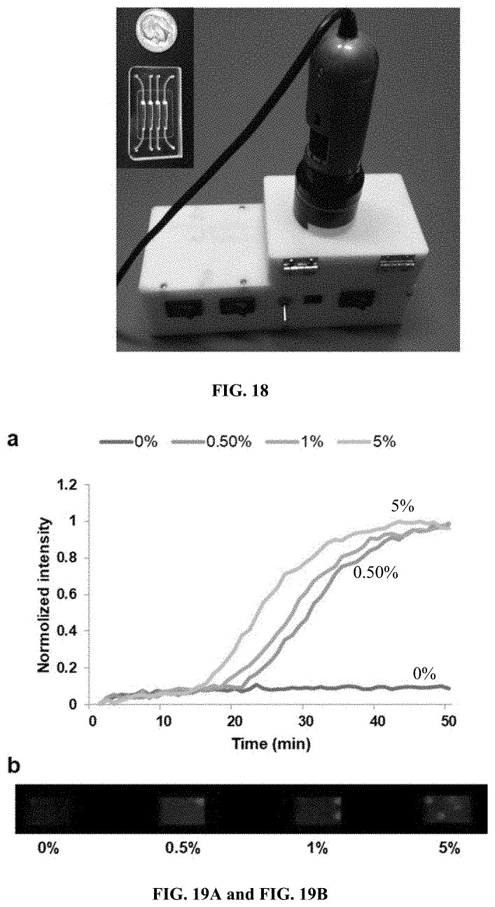

Custom-made microfluidic chips were used with four independent multifunctional, isothermal amplification reactors. For each test, 600-μL mixture of 200-μL plasma, 200-μL Qiagen AL buffer, and 200-μL ethanol was filtered through the nucleic acid isolation membrane of one of the amplification reactors. The nucleic acids bound to the membrane. Subsequent to the sample introduction, 150 μL of Qiagen wash buffer 1 (AW1) was injected into the reactor to remove amplification inhibitors. Then, the silica membrane was washed with 150 μL of Qiagen™ wash buffer 2 (AW2), followed by air-drying for 30 seconds. Next, 25 μl of real-time PASEA reaction mixture prepared as described above was injected into each reactor. The inlet and outlet ports were then sealed with transparent tape. The chip was placed in a portable custom-made device (Kadimisetty et al., Biosensors and Bioelectronics, 109, 156-163, 2018) that houses a heating system and USB-based microscope (FIG. 18) for fluorescence excitation and emission imaging.

Results

PASEA Enables High Sensitivity Detection with Inexpensive (Sanger) Sequencing

RNP is Compatible with RPA.

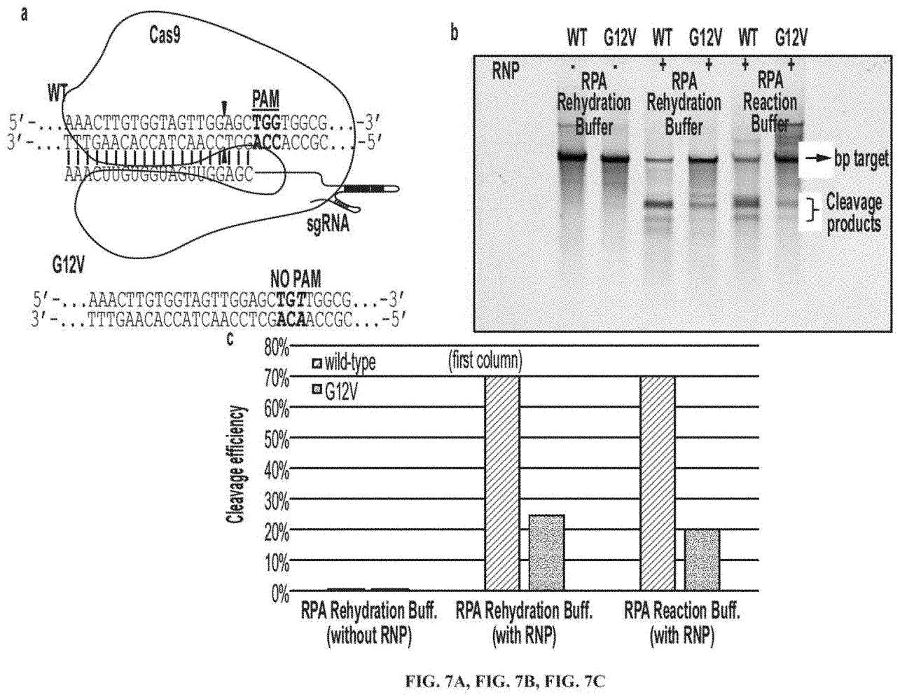

To verify that CRISPR-Cas9 is compatible with RPA, ribonucleoprotein (RNP, Cas9 protein combined with the targeting sgRNA) was added to the RPA reaction buffer in the absence of primers. During one-hour incubation (without amplification), the assay cleaved nearly 70% of the 100 bp WT synthetic dsDNA KRAS that contains the PAM site and 20% of the G12V, in which the PAM site is absent (FIG. 7A), indicating that RNP functions effectively when in the RPA reaction mix.

PASEA Enriches Exponentially Mutant Allele Fraction (MA).

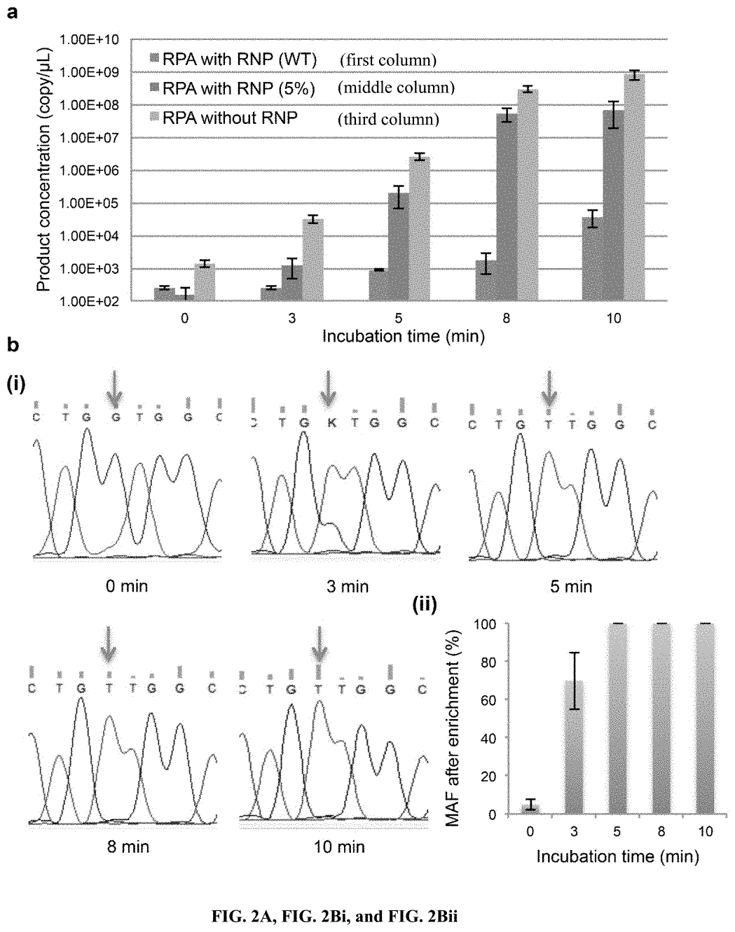

Pure WT-KRAS dsDNA and blends containing KRAS G12V dsDNA (MAF 5%) were incubated with PASEA (0.1 μM RNP) for various time spans along with a control subjected to RPA without RNP. The products were quantified with a pre-calibrated qPCR (FIG. 8). As the incubation time increased, the number of amplicons in both the pure and mixed samples increased, with the number of amplicons in the mixed sample increasing much faster than in the pure sample (FIG. 2A). After a 5 min PASEA incubation, the number of amplicons in the mixed sample was two orders of magnitude larger than that in the pure WT sample. The mutant allele was preferentially amplified, and the MAF increased from 5% to nearly 100% as the incubation time increased (FIG. 2Bii).

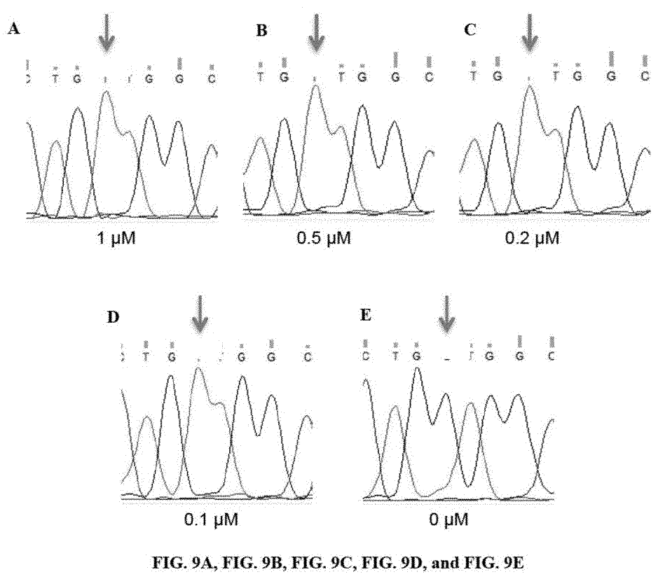

To verify that the amplicons are, indeed KRAS-G12V, the PASEA products of 60 ng genomic DNA with 5% KRAS G12V were subjected to Sanger sequencing (FIG. 2Bi). The presence of (5%) mutant allele in the sample was barely visible in the absence of PASEA; appeared clearly after 3 min of incubation; and dominated the signal after 5 min and longer incubation, the MAFs was estimated with Mutation Surveyor Software (https://softgenetics.com/mutationSurveyor.php) (FIG. 2Bii). Consistent with the qPCR results, the Sanger sequencing data show that the MAF has increased from 5% to 70% after 3 min PASEA incubation and to nearly 100% after 5 min or longer incubation. PASEA provides highly efficient enrichment with RNP concentrations ranging from 0.1 μM to 1 μM (FIGS. 9A-9D).

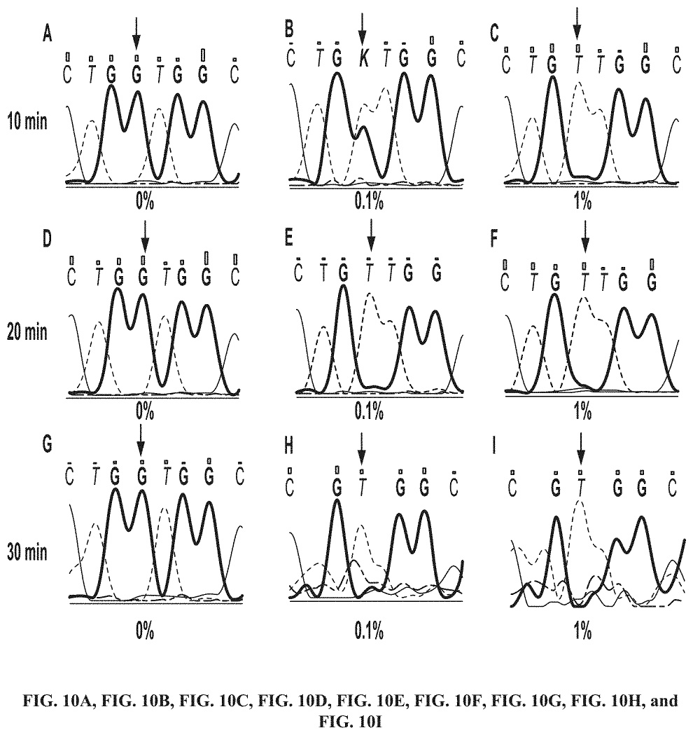

The optimal incubation time depends on the sample's MAF (FIGS. 10A-10I). When MAF=1%, incubation time of 10 min is sufficient to deplete the WT to undetectable level in the Sanger sensorgram. When MAF=0.1%, both the WT and MA signatures are evident in the sensorgram after 10 min PASEA. Only the MA is evident after 20 min PASEA. Hence, in most of the experiments that follow, 20 min incubation time was used. Longer incubation times such as 30 min are undesirable as they result in noisy sensorgrams possibly due to the presence of spurious amplicons.

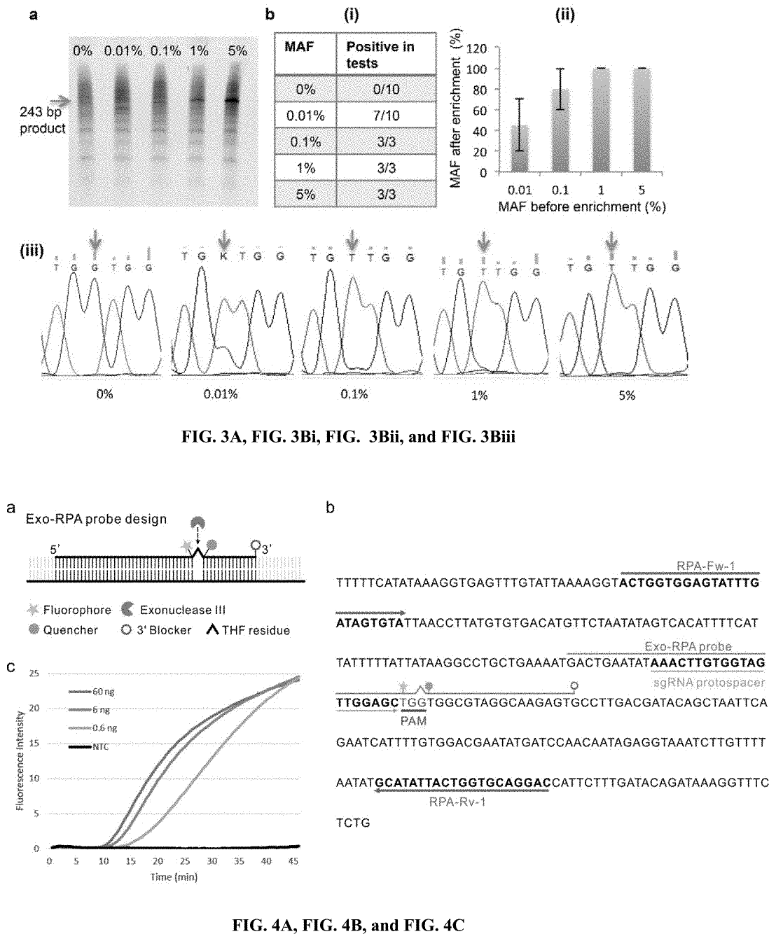

A standard genomic DNA panel with 0%, 0.01%, 0.1%, 1%, and 5% KRAS G12V MAFs was subjected to 20 min PASEA incubation and examined the incubation products with gel electrophoresis and Sanger sequencing (FIGS. 3A and 3Bi-3Biii). Electropherograms bands of PASEA products of samples with 5% and 1% MAF were clearly visible at 243 bp (FIG. 3A), indicating that the KRAS G12V mutant genomic DNA was successfully enriched. Sanger sequencing (FIG. 3B-i) identified the presence of KRAS G12V in all PASEA products of samples with MAF=0.1%, 1%, or 5% (N=3) and in 70% of the PASEA products of samples with MAF=0.01% (N 10). PASEA increased the MAFs to nearly 100% in the 5% (20-fold enrichment) and 1% (100-fold enrichment) samples; to 80% (800-fold enrichment) in the 0.1% sample; and to 40% (4000-fold enrichment) in the 0.01% samples (FIG. 3B-ii). The less than perfect performance with the 0.01% MAF samples may be attributed to a sampling error due to the scarcity of mutant alleles in these samples. PASEA did not produce any false positives.

Real-Time PASEA

PASEA Products are Detectable in Real Lime

PASEA in combination with Sanger sequencing, qPCR, or polyacrylamide gel electrophoresis (PAGE) provides sensitive, two-stage detection of rare alleles. To meet the needs of resource poor settings, a single stage, closed pot assay was designed. The real time assay uses an Exo-RPA probe (FIG. 4A) comprised of an abasic nucleotide analogue (tetrahydrofuan residue, THF) with a flanking dT-fluorophore at one end, a dT-quencher on the other, and a C3-spacer to block polymerase extension. When free in solution, the probe's fluorophore is quenched by the quencher located 2-5 bases away from the fluorophore. The Exonuclease III enzyme (included in the TwistAmp® exo kit) digests the probe when it hybridizes with the amplicon to form a double-strand context and separates the fluorophore from the quencher, enabling fluorescent emission.

The short target (˜160 bp)(20, 21) challenges probe design. It is difficult to avoid an overlap between hybridization sites for the probe and the sgRNA. Several Exo-RPA probe sequences were designed and evaluated (FIGS. 11A-11F) and selected the best performer (FIG. 4B) that hybridizes to the amplicon's middle region. Since the THF localizes to the KRAS G12V/D location, the Exo-RPA probe does not discriminate between the WT and mutant alleles, but instead reports on the total number of amplicons (FIG. 4C).

High Probe Concentration Interferes with PASEA

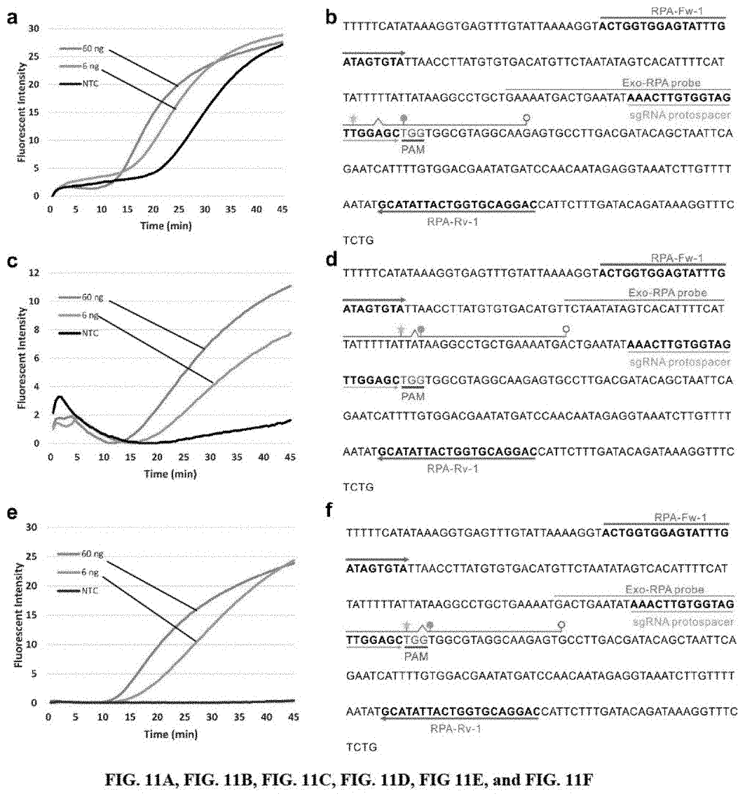

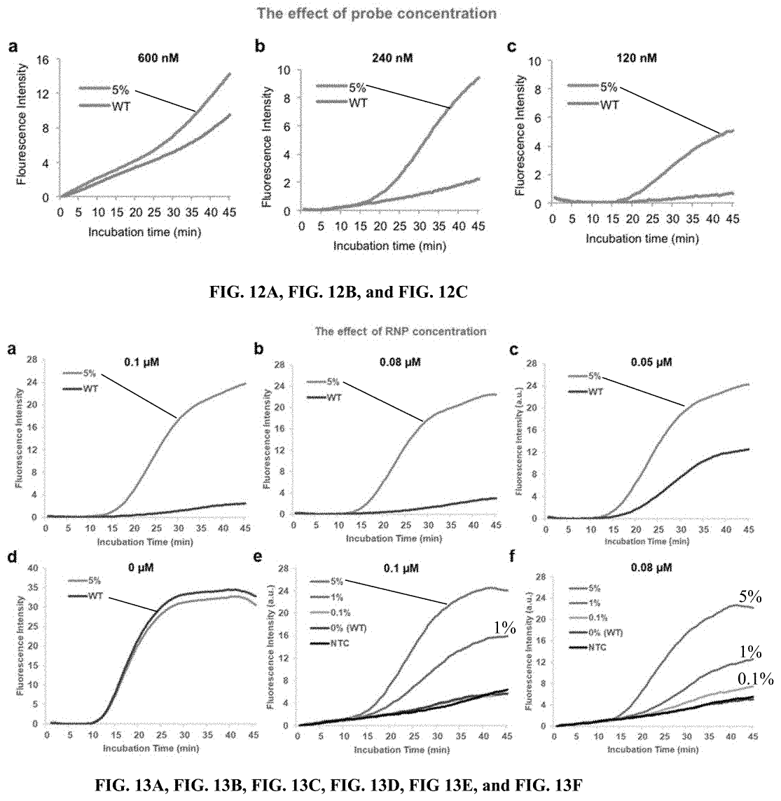

Since the probe and the gRNA target have overlapping sequences, it is necessary to minimize the probe's effect on PASEA cleaving efficiency (FIGS. 12A-12C). At probe concentrations of 120 and 240 nM, PASEA discriminates well between 5% KRAS G12V and WT alleles while at higher probe concentrations (e.g., 600 nM), there is little contrast between 5% KRAS G12V and WT alleles; likely because of probe interference with the sgRNA hybridization and insufficient Exonuclease III to digest excess Exo-RPA probes. In all the subsequent experiments, 240 nM probe concentration was used that provided a brighter signal than the 120 nM concentration, therefore reducing demands on the imaging system.

Optimal RNP Concentration for Real-Time PASEA

Considering the interference between the probe and the RNP, the effect of RNP concentration on the real time amplification curve in the presence of 240 μM probe was examined (FIGS. 13A-13F). Reaction mixes with 0.1 (a) and 0.08 μM (b) RNP discriminated well between samples of 0% and 5% KRAS G12V gDNA while 0.05 μM RNP (c) provided less satisfactory contrast. To further fine tune the assay for cfDNA detection, the effect of RNP concentrations on real time PASEA acting on a standard KRAS G12V cfDNA control (0%. 0.1%, 1%, and 5% MAF) were compared. The assay with 0.08 μM RNP rendered 0.1% MAF (FIG. 13F) detectable while the same MAF was not detectable with 0.1 μM RNP.

Real-Time PASEA Co-Detects ctDNA and ctRNA

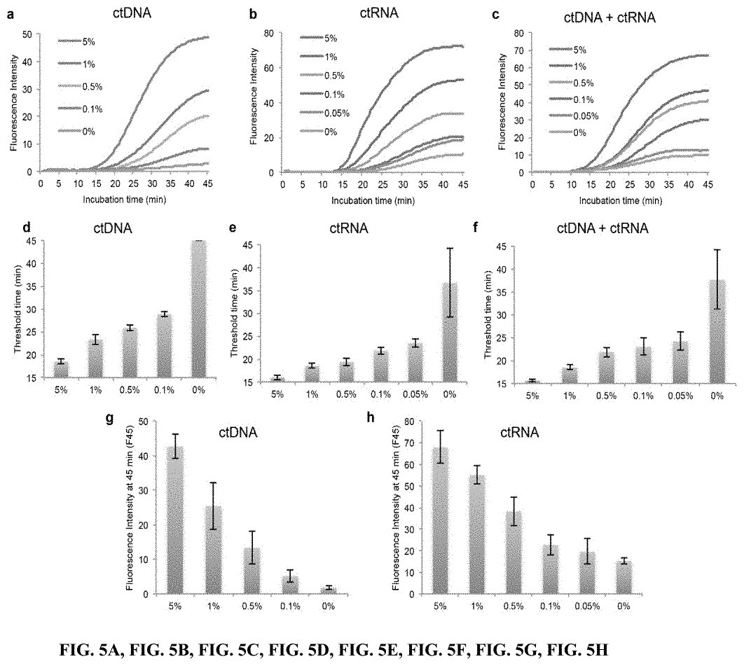

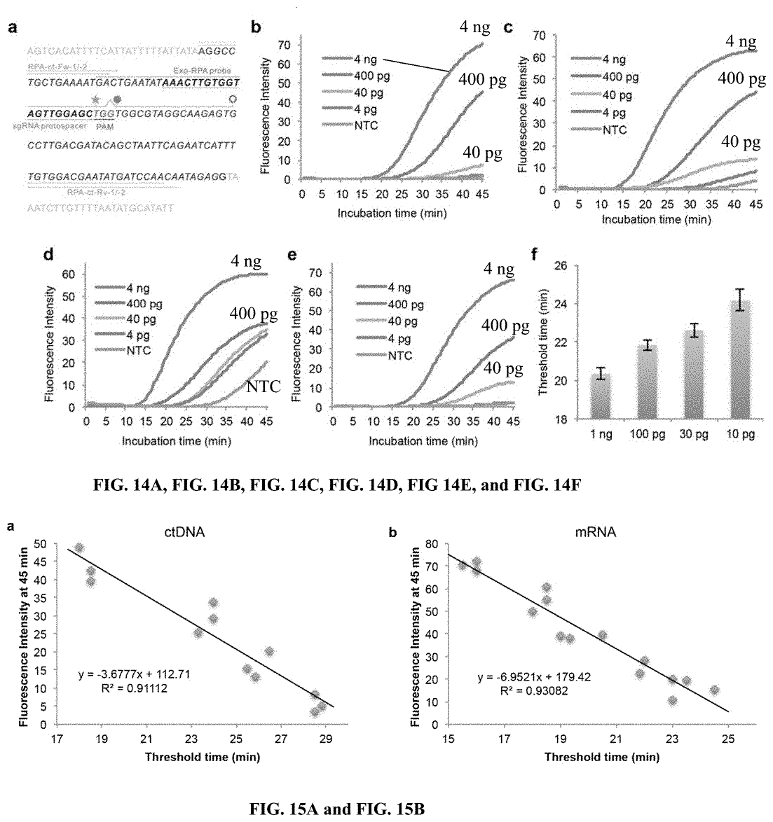

At early disease stages, cell free mutant alleles are present in body fluids at very low concentrations. cfRNA (predominantly, small RNAs and mRNAs) is packaged in exosomes, present in peripheral blood, and protected from degradation (22, 23). To increase the number of biomarkers, cell free, tumor derived fragments of both DNA and RNA were target (24). Since the ctDNA and the KRAS exon 2 (122 nt) that share the common sequence (125 nt, FIG. 14A) are short (˜160 nt), various primers for short amplicons were designed and tested (FIG. 14A and Table 1) to concurrently amplify both ctDNA and complementary DNA. RAS(G12D was targeted, which like KRAS G12V, lacks the NGG PAM. Real time PASEA with primers RPA-ct-Fw-1/RPA-ct-Rv-2 detects KRAS G12D in 20 ng of standard cfDNA in the absence of reverse transcriptase (RT) (FIGS. 5A, 5D); in 400 ng of purified mRNA (˜120,000 copies of the target) in the presence of RT (FIGS. 5B, 5E); and in a mixture of 10 ng cfDNA and 200 ng mRNA in the presence of RT (FIGS. 5C, 5F) at various MAFs. These nucleic acid masses are comparable with patient samples. As expected, the threshold time (defined as the time until signal intensity exceeds 10% saturation level) increases as the MAF decreases (FIGS. 5D-5F). PASEA readily detects 0.1% MAF cfDNA (˜6 copies in 20 ng, FIGS. 5A, 5D), 0.05% G12D mRNA (˜60 copies in 400 ng, FIGS. 5B, 5E) and 0.05% mixture of ctDNA and mRNA (FIGS. 5C, SF).

Interestingly, the fluorescence intensity of the amplification curve's plateau increases as the MAF increases. The fluorescence intensity of the plateau at 45 min (F₄₅) correlates well with the MAF (FIGS. 5G, 5H) and with the threshold time (FIGS. 15A and 15B), providing yet another metric for semi-quantitative estimation of the MAF.

Real Time PASEA of Clinical Samples Concords with NGS and ARMS-PCR

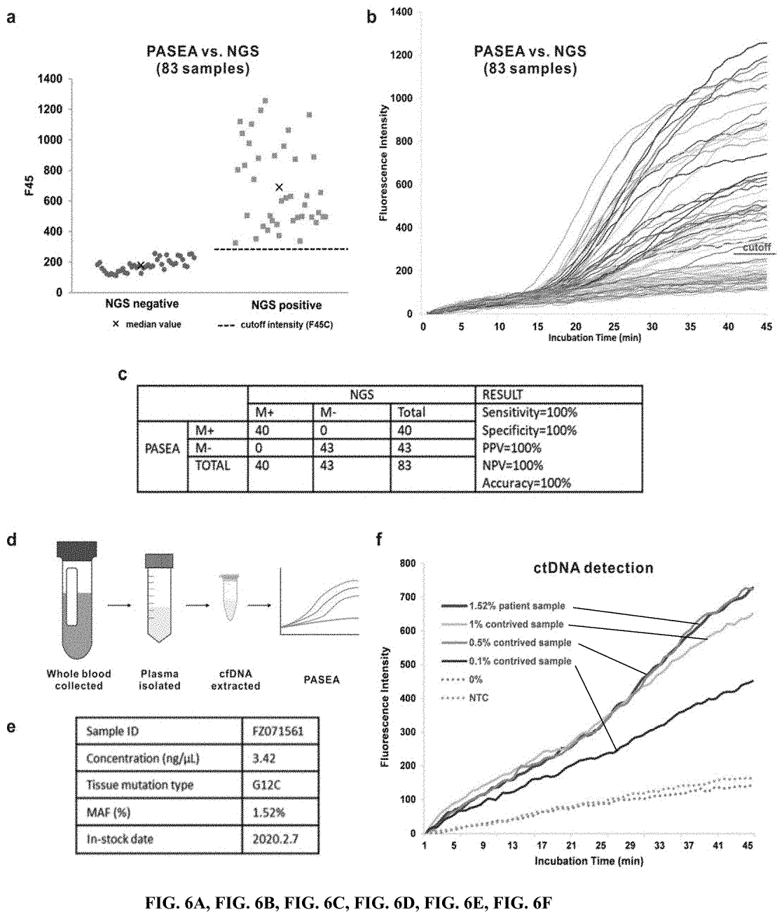

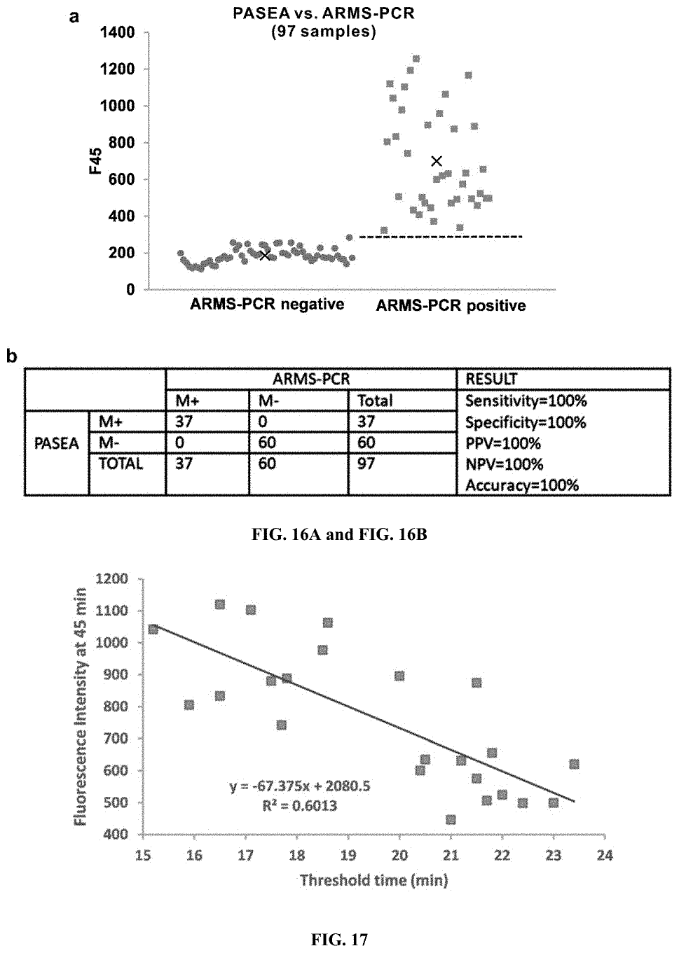

Tissue samples from 108 colorectal cancer (62/108), pancreatic cancer (1/108), and lung cancer patients (45/108) were collected by either resection or biopsy at the Cancer Hospital of the Chinese Academy of Medical Sciences (Beijing, China). Genomic DNA (gDNA) was extracted from these tissue samples and tested with clinical NGS (83 samples) and ARMS-PCR (97 samples). 40 samples were positive to KRAS mutations (G12V, D, S. C, and R) with MAF ranging from 1% to 39%. 68 samples were negative for KRAS mutations (Table 2). The extracted gDNAs were diluted to 10 ng/μL and then tested with real-time PASEA. Real-time PASEA was deemed positive when F₄₅ exceeded the cutoff (F₄₅C), defined as the average F₄₅ plus 3 SD (95% confidence level) for standard WT gDNA at 20 ng/μL, which is greater than the DNA concentration in the clinical samples (10 ng/μL) (Table 2). Real-time PASEA correctly identified 40/40 samples as positive and 68/68 samples as negative (FIGS. 6A-6F), exhibiting 100% sensitivity, specificity, positive predictive value, negative predictive value, and concordance with NGS and/or ARMS-PCR genotyping for KRAS G12 mutations (FIGS. 6A-6C and FIGS. 16A, 168). The emission intensity (F₄₅) correlates well with the threshold time (FIG. 17).

TABLE 2
Genomic DNA extracted from 108 tissue samples (29 patients diagnosed with colonic
adenocarcinoma, 33 patients diagnosed with rectal adenocarcinoma, 45 patients
diagnosed with lung cancer, and 1 patient diagnosed with pancreatic cancer).

| Blinded | | | Tissue | | ARMS- | |
|---|---|---|---|---|---|---|
| sample | | Tissue | mutation | | PGR | |
| No. | Diagnosis | resource | type | NGS* | (KRAS)* | PASEA |
| 1 | colonic | resection | WT | N | | N |
| | rectal adenocarcinoma | | | | | |
| 2 | rectal adenocarcinoma | resection | WT | N | N | N |
| 3 | colonic | resection | WT | N | N | N |
| | adenocarcinoma | | | | | |
| 4 | rectal adenocarcinoma | biopsy | WT | N | N | N |
| 5 | rectal adenocarcinoma | resection | WT | N | N | N |
| 6 | rectal adenocarcinoma | resection | WT | N | N | N |
| 7 | rectal adenocarcinoma | resection | WT | N | N | N |
| 8 | rectal adenocarcinoma | resection | WT | N | N | N |
| 9 | rectal adenocarcinoma | resection | WT | N | N | N |
| 10 | rectal adenocarcinoma | resection | WT | N | N | N |
| 11 | colonic | resection | WT | N | N | N |
| | adenocarcinoma | | | | | |
| 12 | colonic | resection | WT | N | N | N |
| | adenocarcinoma | | | | | |
| 13 | rectal adenocarcinoma | resection | WT | N | N | N |
| 14 | colonic | resection | WT | N | N | N |
| | adenocarcinoma | | | | | |
| 15 | lung cancer | resection | WT | N | | N |
| 16 | lung cancer | resection | WT | N | | N |
| 17 | lung cancer | resection | WT | N | | N |
| 18 | lung cancer | resection | WT | N | | N |
| 19 | lung cancer | resection | WT | N | | N |
| 20 | lung cancer | resection | WT | N | | N |
| 21 | colonic | resection | G12C | P | P | P (+) |
| | adenocarcinoma | | | | | |
| 22 | colonic | resection | G12D | P | P | P (+++) |
| | adenocarcinoma | | | | | |
| 23 | colonic | resection | G12D | P | P | P (++++) |
| | adenocarcinoma | | | | | |
| 24 | colonic | resection | G12D | P | P | P (++++) |
| | adenocarcinoma | | | | | |
| 25 | colonic | biopsy | G12D | P | P | P (+++) |
| | adenocarcinoma | | | | | |
| 26 | colonic | resection | G12D | P | P | P (+) |
| | adenocarcinoma | | | | | |
| 27 | colonic | resection | G12D | P | P | P (+++) |
| | adenocarcinoma | | | | | |
| 28 | rectal adenocarcinoma | biopsy | G12V | P | P | P (++++) |
| 29 | rectal adenocarcinoma | resection | G12D | P | P | P (++) |
| 30 | lung cancer | resection | G12V | P | | P (+) |
| 31 | lung cancer | resection | G12C | P | | P (+++) |
| 32 | rectal adenocarcinoma | resection | G12V | P | P | P (++++) |
| 33 | rectal adenocarcinoma | resection | G12S | P | P | P (+) |
| 34 | colonic | biopsy | G12V | P | P | P (++++) |
| | adenocarcinoma | | | | | |
| 35 | rectal adenocarcinoma | resection | G12V | P | P | P (+) |
| 36 | rectal adenocarcinoma | resection | G12V | P | P | P (+) |
| 37 | rectal adenocarcinoma | resection | G12V | P | P | P (+) |
| 38 | rectal adenocarcinoma | resection | G12C | P | P | P (+++) |
| 39 | pancreatic cancer | resection | G12V | P | P | P (+) |
| 40 | rectal adenocarcinoma | resection | G12D | P | P | P (+) |
| 41 | colonic | biopsy | G12V | P | P | P (++) |
| | adenocarcinoma | | | | | |
| 42 | colonic | biopsy | G12V | P | P | P (+++) |
| | adenocarcinoma | | | | | |
| 43 | rectal adenocarcinoma | resection | G12D | P | P | P (++) |
| 44 | colonic | resection | G12D | P | P | P (++++) |
| | adenocarcinoma | | | | | |
| 45 | colonic | resection | G12D | P | P | P (++) |
| | adenocarcinoma | | | | | |
| 46 | rectal adenocarcinoma | biopsy | G12D | P | P | P (+) |
| 47 | rectal adenocarcinoma | resection | G12C | P | P | P (++) |
| 48 | colonic | resection | G12C | P | P | P (+) |
| | adenocarcinoma | | | | | |
| 49 | rectal adenocarcinoma | resection | G12S | P | P | P (+) |
| 50 | lung cancer | resection | G12C | P | | P (+) |
| 51 | lung cancer | resection | G12C | P | P | p (++) |
| 52 | lung cancer | resection | G12V | P | P | P (++) |
| 53 | lung cancer | resection | G12D | P | P | P (++++) |
| 54 | lung cancer | resection | G12C | P | P | P (+) |
| 55 | lung cancer | resection | G12V | P | P | P (+++) |
| 56 | lung cancer | resection | G12R | P | P | P (+) |
| 57 | lung cancer | resection | G12C | P | P | P (++) |
| 58 | lung cancer | resection | G12D | P | P | p (++) |
| 59 | lung cancer | resection | G12D | P | P | P (+) |
| 60 | lung cancer | resection | G12V | P | P | P (+) |
| 61 | colonic | resection | WT | N | N | N |
| | adenocarcinoma | | | | | |
| 62 | rectal adenocarcinoma | resection | WT | N | N | N |
| 63 | rectal adenocarcinoma | resection | WT | N | N | N |
| 64 | colonic | resection | WT | N | N | N |
| | adenocarcinoma | | | | | |
| 65 | rectal adenocarcinoma | biopsy | WT | N | N | N |
| 66 | rectal adenocarcinoma | resection | WT | N | N | N |
| 67 | colonic | resection | WT | N | N | N |
| | adenocareinoma | | | | | |
| 68 | rectal adenocarcinoma | resection | WT | N | N | N |
| 69 | rectal adenocarcinoma | resection | WT | N | N | N |
| 70 | rectal adenocarcinoma | resection | WT | N | N | N |
| 71 | rectal adenocarcinoma | resection | WI | N | N | N |
| 72 | colonic | resection | WT | N | N | N |
| | adenocarcinoma | | | | | |
| 73 | colonic | resection | WT | N | N | N |
| | adenocarcinoma | | | | | |
| 74 | colonic | biopsy | WT | N | N | N |
| | adenocarcinoma | | | | | |
| 75 | rectal adenocarcinoma | biopsy | WT | N | N | N |
| 76 | colonic | resection | WT | N | N | N |
| | adenocarcinoma | | | | | |
| 77 | colonic | resection | WT | N | N | N |
| | adenocarcinoma | | | | | |
| 78 | colonic | biopsy | WT | N | N | N |
| | adenocarcinoma | | | | | |
| 79 | colonic | resection | WT | N | N | N |
| | adenocarcinoma | | | | | |
| 80 | rectal adenocarcinoma | resection | WT | N | N | N |
| 81 | rectal adenocarcinoma | resection | WT | N | N | N |
| 82 | colonic | resection | WT | N | N | N |
| | adenocarcinoma | | | | | |
| 83 | lung cancer | resection | WT | | N | N |
| 84 | lung cancer | resection | WT | | N | N |
| 85 | lung cancer | resection | WT | | N | N |
| 86 | lung cancer | resection | WT | | N | N |
| 87 | lung cancer | resection | WT | | N | N |
| 88 | lung cancer | resection | WT | | N | N |
| 89 | lung cancer | resection | WT | | N | N |
| 90 | lung cancer | resection | WT | | N | N |
| 9i | lung cancer | resection | WT | | N | N |
| 92 | lung cancer | resection | WT | | N | N |
| 93 | lung cancer | resection | WT | | N | N |
| 94 | lung cancer | resection | WT | | N | N |
| 95 | lung cancer | resection | WT | N | | N |
| 96 | lung cancer | resection | WT | | N | N |
| 97 | lung cancer | resection | WT | | N | N |
| 98 | lung cancer | resection | WT | | N | N |
| 99 | lung cancer | resection | WT | | N | N |
| 100 | lung cancer | resection | WT | | N | N |
| 101 | lung cancer | resection | WT | | N | N |
| 102 | lung cancer | resection | WT | | N | N |
| 103 | lung cancer | resection | WT | | N | N |
| 104 | lung cancer | resection | WT | | N | N |
| 105 | lung cancer | resection | WT | | N | N |
| 106 | lung cancer | resection | WT | | N | N |
| 107 | lung cancer | resection | WT | | N | N |
| 108 | lung cancer | resection | WT | | N | N |
| Positive | | | G12V | | | P (++++) |
| control | | | (33%) | | | |
| Positive | | | G12V | | | P (++) |
| control | | | (5%) | | | |
| Positive | | | G12V | | | P (+) |
| control | | | (1%) | | | |

“+++++”, “+++”, “++”, “+” indicates, respectively, positive when F₄₅ >1006.8, >756.8, >450.0, >285.7
Cutoff (0%) = F₄₅ + 3SD (N = 6) = 181.9 + 3 × 34.6 = 285.7
*From the 108 tissue biopsy and resection samples, 83 were tested with NGS, 97 were tested with ARMS-PCR (KRAS), and 72 were tested with both methods.

Plasma samples were collected from 10 lung cancer patients and banked at −80° C. NGS identified only one sample as positive for KRAS mutation (KRAS G12C, MAF 1.52%, FIG. 6E). To compensate for the small number of positive clinical samples. Horizon standard cfDNA (WT) controls were spiked into the positive sample to form contrived samples with MAFs of 1%, 0.5%, and 0.1%. The 10 patient samples and the 3 contrived samples were subjected to real-time PASEA. The amplification curves of positive samples clearly differentiated from the curve of the WT control (0%) (FIG. 6F), indicating that real time PASEA detects ctDNA in blood samples with high sensitivity.

PASEA can be Implemented in Microfluidic Chip for Use at the Point of Need

Real-time PASEA is relatively easy to carry out, does not require strict temperature control, and can be used at the point of care (25). Real-time PASEA was implemented in a multifunctional isothermal amplification microfluidic (MIAR) chip (25) that extracts and concentrates nucleic acids from a sample and mates with a portable isothermal amplification processor (26) (FIG. 18). PASEA was carried out on samples of various concentrations of standard KRAS WT cfDNA and G12D ctDNA spiked in PBS. Samples with MAF of 0.5% of KR4S G12D were readily detected within 40 min (FIGS. 19A and 19B). Real time PASEA exhibited a detection limit of about 87 copies when operating with samples of 60 ng cfDNA.

Discussion

Researchers have identified various oncogenic mutations responsible for the initiation and maintenance of cancer and the mechanisms of resistance to targeted therapeutics (27), enabling (A) effective, targeted, genotype-specific therapies; (B) avoidance of the use of ineffective, potentially harmful drugs: and (C) monitoring therapy's efficacy and evolution of drug-resistance. For example, KRAS mutations are, respectively, present in ˜8.3% (28) and ˜40% (29) of lung and colorectal cancer patients, and in even a greater percentage in metastatic patients. Methods for cost effective, non-invasive cancer genotyping are needed to enable targeted therapies. An attractive genotyping method relies on identifying cell-free, tumor derived, aberrant nucleic acids in body fluids (liquid biopsy). However, the identification of tumor-associated nucleic acid fragments in body fluids is challenged by their low abundance and sequence homology with the vast background of nucleic acids from healthy cells.

MAF enrichment is essential for timely, sensitive detection of clinically critical, rare mutant alleles. Such an enrichment can be accomplished by hybridizing nucleic acids of interest to nucleic acid probes in the absence or presence of nucleic-acid guided endonucleases lacking catalytic activity (e.g., dCas9) (30, 31); by preferential enzymatic amplification of mutant alleles with specifically designed primers and DNA blocker (1, 2, 5); by suppression of the amplification of wild type alleles with capping nucleic acids (3); and by selective depletion of wild type nucleic acids with programmable endonuclease such as CRISPR Cas 9 (6, 19) and Argnoautes (7), wherein unwanted background sequences are selectively removed from the sample. These various methods can be used independently or in combination. The targeted nucleic acids can then be detected either directly or by sequencing.

Among the aforementioned methods, the CRISPR-mediated, Ultrasensitive detection of Target DNA by PCR (CUT-PCR) (19) is a promising method to enrich mutant alleles' fraction by rounds of selective depletion of WT. The Cas9-mediated depletion of WT alleles and downstream PCR are usually repeated 2 or 3 times to achieve desired sensitivity. CUT-PCR has successfully increased MA F by 27-fold in most samples with 0.1% MAF (19). In combination with deep sequencing, CUT-PCR enables detection of 0.01% mutant alleles.

There are, however, a few factors that limit the enrichment level achievable with cleaving assays such as CUT-PCR. Although Cas 9 preferentially cleaves WT alleles, it also cleaves, albeit, to a lesser degree off-target mutant alleles. While off-target and target cleavage rates depend on the guide RNA design. Cas9 variant, and assay conditions, samples with low MAF (e.g., 0.01%) contain just a handful of molecules of interest and any loss of biomarkers would compromise assay's sensitivity. Furthermore, not all guide protein-target triplexes are productive. By some estimates, fewer than 90% triplexes are cleaved (32). Since the dissociation rate of the triplex is very slow, a significant fraction of WT DNA is protected from cleavage but amenable to PCR amplification. For example, if an assay cleaves only 90% of the wild type alleles, the MAF can be enriched by less than 10-fold in a single step which is consistent with the 27-fold enrichment achieved by CUT in three cleavage-PCR rounds.

Inspired by Darwin theory of the survival of the fittest, a simple remedy that combines cleavage with concurrent polymerase amplification to overcome the shortcomings of the cleavage only assays is presented herein. The assay amplifies concurrently mutant alleles of interest and WT alleles in the presence of relatively high concentration of guided endonuclease. The endonuclease in the system plays the role of a predator while the nucleic acids are the prey with the WT alleles much more vulnerable than the mutant alleles. While the copy numbers of both the WT and mutant allele increase with time, the latter do so at much greater rate, alleviating any concerns of losing valuable biomarkers.

PASEA exhibited high sensitivity (FIGS. 3A, 3Bi-3Biii), enabling Sanger sequencing of samples with MAF>0.1% and many (70%) of the samples with MAF ˜0.01% after PASEA incubation. 60 ng of DNA was used in the experiments, which corresponds to approximately 17,400 copies of nucleic acids. MAF f_{MA}=0.01% is equivalent to ˜2 copies of mutant allele per sample. This shows that PASEA enabled enrichment and detection of as few as 2 copies of mutant allele in a sample and that the 70% sensitivity observed for f_{MA}=0.01% results from sampling error since a few of the samples may not have contained any mutant alleles, and not from any PASEA deficiency.

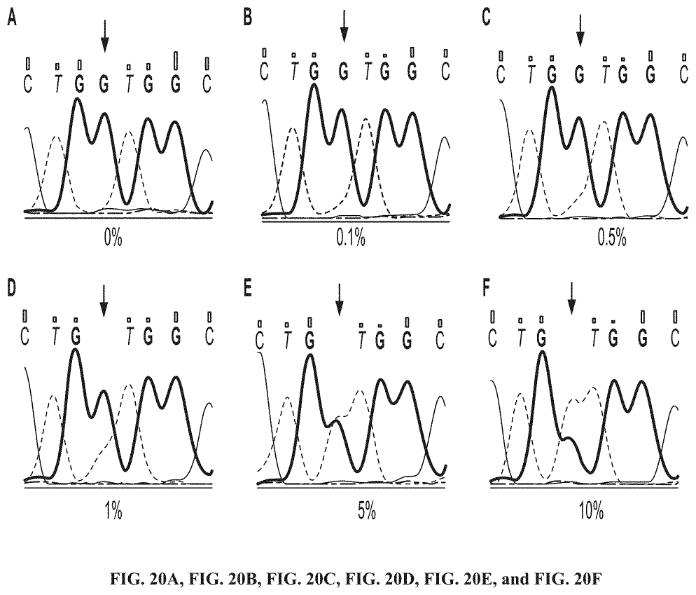

How does PASEA compare to other Cas9-based cleaving assays (e.g. DASH)? The samples were incubated with DASH for 20 min and analyzed the products with Sanger sequencing (FIGS. 20A-20F). DASH enabled detection of mutant alleles only in samples with a MAF>1% (FIGS. 20A-20F) and under optimal conditions with a MAF>0.1% (6) (Table 3). The newly developed PASEA method has about two orders of magnitude better performance than DASH in terms of the lowest detectable MAR.

TABLE 3
Comparison of various Cas9-assisted mutant allele enrichment methods

| MAF before | | Incubation time | MAF after | |
|---|---|---|---|---|
| enrichment | Method | (min) | enrichment | Fold enrichment |
| 0.01% | DASH(5) | N/A | N/A | N/A |
| | CUT-PCR(6) | 60 | 0.35% | 18 |
| | PASEA | 20 | 40% | 4000 |
| 0.1% | DASH(5) | 60 | 6.5% | 65 |
| | CUT-PCR(6) | 60 | 3% | 30 |
| | PASEA | 20 | 80% | 800 |
| 1% | DASH(5) | 60 | 30% | 30 |
| | CUT-PCR(6) | 60 | 20% | 20 |
| | PASEA | 20 | 100% | 100 |

(5) Gu et al., Genome Biology 2016;17 1:41;
(6) Lee et al., Oncogene 2017 Dec. 7;36 49:6823-9, Epub 2017 Aug. 29 as doi: 10,1038/onc.2017,281

PASEA's very high enrichment capability enables preparing libraries for rapid, low-cost sequencers such as Sanger (FIGS. 3A, 3Bi-3Biii). It also offers the opportunity for real time detection of mutant alleles in a closed pot without a sequencer, eliminating the need to open amplicon-rich tubes and risking the contamination of the workspace. The real time assay (FIGS. 5A-5H) successfully identified the presence of mutant alleles in samples with MAF of less than 0.1% of both DNA and RNA. PASEA is semi-quantitative. The number of the mutant allele copies can be estimated either from the threshold time or the fluorescence emission intensity after the signal has plateaued—at 45 min after the start of PASEA. The emission intensity (F₄₅) and the threshold time correlate well (FIGS. 15A, 15B and 17).

Clinical evaluation of PASEA exhibits 100% sensitivity, specificity, positive predictive value, negative predictive value, and concordance with NGS and ARMS-PCR genotyping for KRAS G12 mutations when testing tissue samples. PASEA also has successfully identified the presence of mutant alleles in all positive samples and yielded no false positives. PASEA enables an unprecedented level of enrichment and detection with relatively simple instruments, providing effective manners for cancer screening and targeted therapies in low resource settings.

PASEA's reliance on Cas9-like proteins limits its use to sequences in which the PAM motif is present in the wild type allele and absent in the mutant allele. Lee et al. (19) estimate, however, that with the use of various orthologonal CRISPR endonucleases such as SpCas9 and FnCpf1, Cas9-like proteins can target about 80% of known cancer-linked substitution mutations registered in the Catalogue of Somatic Mutations in Cancer (COSMIC) database.

### REFERENCES

1. Newton C R, Graham A, Heptinstall L E, Powell S J, Summers C, Kalsheker N, Smith J C, et al. Analysis of any point mutation in DNA. The amplification refractory mutation system (ARMS). Nucleic Acids Res 1989 Apr. 11; 17 7:2503-16. Epub 1989/04/11 as doi: 10.1093/nar/17.7.2503.
2. Wu L R, Chen S X, Wu Y, Patel A A, Zhang D Y. Multiplexed enrichment of rare DNA variants via sequence-selective and temperature-robust amplification. Nat Biomed Eng 2017:1:714-23. Epub 2017/01/01 as doi: 10.1038/s41551-017-0126-5.
3. Narumi S, Matsuo K, Ishii T, Tanahashi Y, Hasegawa T. Quantitative and sensitive detection of GNAS mutations causing mocune-albright syndrome with next generation sequencing. Plos One 2013; 8 3:e60525. Epub 2013/03/29 as doi: 10.1371/journal.pone.0060525.
4. Li J, Wang L, Mamon H, Kulke M H, Berbeco R, Makrigiorgos G M. Replacing PCR with COLD-PCR enriches variant DNA sequences and redefines the sensitivity of genetic testing. Nat Med 2008 May; 14 5:579-84. Epub 2008/04115 as doi: 10.1038/nm1708.
5. Vargas D Y, Marras S A E, Tyagi S, Kramer F R. Suppression of Wild-Type Amplification by Selectivity Enhancing Agents in PCR Assays that Utilize SuperSelective Primers for the Detection of Rare Somatic Mutations. J Mol Diagn 2018 July; 20 4:415-27. Epub 2018/04/27 as doi: 10.1016/j.jmoldx.2018.03.004.
6. Gu W, Crawford E D, O'Donovan B D, Wilson M R, Chow E D, Retallack H, Derisi J L. Depletion of Abundant Sequences by Hybridization (DASH): using Cas9 to remove unwanted high-abundance species in sequencing libraries and molecular counting applications. Genome Biology 2016; 17 1:41.
7. Song J, Hegge J W, Mauk M G. Chen J, Till J E, Bhagwat N, Azink L T, et al. Highly specific enrichment of rare nucleic acid fractions using Thermus thermophilus argonaute with applications in cancer diagnostics. Nucleic Acids Res 2020 Feb. 28; 48 4:e19. Epub 2019/12/13 as doi: 10.1093/nar/gkz1165.
8. Haim H. Bau; Jinzhao Song; Changchun Liu; Michael G. Mauk; John van der Oost; Jorrit Hegge; ENRICHMENT OF NUCLEIC ACIDS, PCT/US2019/022255.
9. Sorek R. Kunin V, Hugenholtz P. CRISPR—a widespread system that provides acquired resistance against phages in bacteria and archaea. Nature Reviews Microbiology 2008; 6 3:181-6.
10. Marraffini L A, Sontheimer E J. CRISPR interference: RNA-directed adaptive immunity in bacteria and archaea. Nature Reviews Genetics 2010; 11 3:181-90.
11. Swarts D C, Jore M M, Westra E R, Zhu Y. Janssen J H, Snijders A P, Wang Y. et al. DNA-guided DNA interference by a prokaryotic Argonaute. Nature 2014:507 7491:258-61.
12. Swarts X C. Hegge J W, Ismael H. Masami S. Ellis M A, Justin D, Terns R M, et al, Argonaute of the arcbaeon Pyrococcus furiosus is a DNA-guided nuclease that targets cognate DNA. Nucleic Acids Research 2015 10:10.
13. Chen J S, Ma E, Harrington L B, Costa M D, Tian X, Palefsky J M, Doudna J A. CRISPR-Cas12a target binding unleashes indiscriminate single-stranded DNase activity. Science 2018; 360 6387:436-9.
14. Li S, Cheng Q, Wang J, Li X, Zhang Z, Gao s, Cao R, et al. CRISPR-Cas12a-assisted nucleic acid detection. Cell Discovery 2018; 4 1:20.
15. Gootenberg J S, Abudayyeh O O, Lee J W, Essletzbichler P, Dy A J, Joung J, Verdine V, et al. Nucleic acid detection with CRISPR-Cas13a/C2c2. Science 2017:356 6336:438-42.
16. Freije C A, Myhrvold C, Boehm C K, Lin A E, Sabeti P C. Programmable Inhibition and Detection of RNA Viruses Using Cas13. Molecular Cell 2019; 76 5.
17. Keith, Pardee, Alexander A, Green, Melissa K. Takahashi, Dana, et al. Rapid, Low-Cost Detection of Zika Virus Using Programmable Biomolecular Components. Cell 2016.
18. Sternberg S H, Redding S, Jinek M, Greene E C, Doudna J A. DNA interrogation by the CRISPR RNA-guided endonuclease Cas9. Nature 2014 Mar. 6; 507 7490:62-7. Epub 2014/01/31 as doi: 10.1038/nature13011.
19. Lee S H, Yu J. Hwang G H, Kim S. Kim H S, Ye S. Kim K, et al. CUT-PCR: CRISPR-mediated, ultrasensitive detection of target DNA using PCR. Oncogene 2017 Dec. 7; 36 49:6823-9. Epub 2017/08/29 as doi: 10.1038/onc.2017.281.
20. Underhill H R, Kitzman J O, Sabine H, Welker N C, Riza D, Baker D N, Gligorich K M, et al. Fragment Length of Circulating Tumor DNA. PLoS Genetics 2016; 12 7:e1006162.
21. Sato A. Nakashima C, Abe T, Kato J, Sueoka-Aragane N. Investigation of appropriate pre-analytical procedure for circulating free DNA from liquid biopsy. Oncotarget 2018; 9 61.
22. Heitzer E, Haque I S, Roberts C E S, Speicher M R. Current and future perspectives of liquid biopsies in genomics-driven oncology. Nature Reviews Genetics 2018.
23. Inamdar S, Nitiyanandan R, Rege K. Emerging applications of exosomes in cancer therapeutics and diagnostics. Bioengineering & Translational Medicine 2017; 2 1:70-80.
24. Krug A K, Enderle D, Karlovich C, Priewasser T, Bentink S, Spiel A, Brinkmann K, et al. Improved EGFR mutation detection using combined exosomal RNA and circulating tumor DNA in NSCLC patient plasma [English]. Ann Oncol 2018 March; 29 3:700-6 as doi: 10.1093/annonc/mdx765.
25. Song, Jinzhao, Pandian, Vikram, Mauk, Michael. G., et al. Smartphone-Based Mobile Detection Platform for Molecular Diagnostics and Spatiotemporal Disease Mapping. Analytical chemistry 2018; 90 7:4823-31.
26. Kadimisetty K, Song J, Doto A M, Hwang Y, Peng J, Mauk M G, Bushman F D, et al. Fully 3D Printed Integrated Reactor Array for Point-of-Care Molecular Diagnostics. other 2018; 109.
27. Oxnard G R, Paweletz C P, Kuang Y A, Mach S L, OConnell A, Messineo M M, Luke J J, et al. Noninvasive Detection of Response and Resistance in EGFR-Mutant Lung Cancer Using Quantitative Next-Generation Genotyping of Cell-Free Plasma DNA [English]. Clin Cancer Res 2014 Mar. 15; 20 6:1698-705 as doi: 10.1158/1078-0432.Ccr-13-2482.
28. Zheng D F, Wang R, Zhang Y. Pan Y J, Cheng X H, Cheng C, Zheng S B, et al. The prevalence and prognostic significance of KRAS mutation subtypes in lung adenocarcinomas from Chinese populations [English]. Oncotargets Ther 2016; 9:833-43 as doi: 10.2147/Ott.S96834.
29. Tan C. Du X. KRAS mutation testing in metastatic colorectal cancer. World J Gastroenterol 2012 Oct. 7; 18 37:5171-80. Epub 2012/10/16 as doi: 10.3748/wjg.v18.i37.5171.
30. Guha M, Castellanos-Rizaldos E. Liu P, Mamon H, Makrigiorgos G M. Differential strand separation at critical temperature: a minimally disruptive enrichment method for low-abundance unknown DNA mutations. Nucleic Acids Res 2013 Feb. 1; 41 3:e50. Epub 2012/12/22 as doi: 10.1093/nar/gks1250.
31. Aalipour A, Dudley J C, Park S M, Murty S. Chabon J J, Boyle E A, Diehn M, et al. Deactivated CRISPR Associated Protein 9 for Minor-Allele Enrichment in Cell-Free DNA. Clin Chem 2018 February; 64 2:307-16. Epub 2017/10/19 as doi: 10.1373/clinchem.2017.278911.
32. Yang M, Peng S, Sun R, Lin J, Wang N, Chen C. The Conformational Dynamics of Cas9 Governing DNA Cleavage Are Revealed by Single-Molecule FRET. Cell Rep 2018 Jan. 9; 22 2:372-82. Epub 2018/01/11 as doi: 10.1016/j.celrep.2017.12.048.
33. Hegge J W, Swarts D C. Der Oost J V. Prokaryotic Argonaute proteins: novel genome-editing tools? Nature Reviews Microbiology 2018:16 1:5-11.

What is claimed:

1. A method of selective amplification of a target nucleic acid in a sample, comprising:
amplifying the target nucleic acid in an amplification reaction comprising:
a) the target nucleic acid;
b) a non-target nucleic acid comprising protospacer adjacent motif (PAM);
b) a guide nucleic acid comprising protospacer target sequence that forms a guide/non-target hybrid with the non-target nucleic acid;
d) an endonuclease having an affinity for the guide/non-target hybrid; and
e) a polymerase;
wherein amplifying is amplifying with the polymerase for up to about 120 min, and
wherein the target nucleic acid in the sample is present at a frequency of about 0.001% of nucleic acids in the sample or greater.

2. The method of claim 1, wherein the target nucleic acid is at a frequency between about 0.001% and about 15% of combined target and non-target nucleic acids in the sample, and wherein the frequency of the target nucleic acid is increased between 10 and 10,000 fold.

3. The method of claim 1, wherein the target nucleic acid does not comprise PAM.

4. The method of claim 1, wherein amplifying the target nucleic acid comprises polymerase chain reaction (PCR), loop-mediated isothermal amplification (LAMP), recombinase polymerase amplification (RPA), or any combination thereof, and wherein amplification reaction products provide a library of amplicons for sequencing.

5. The method of claim 1, wherein the target nucleic acid comprises a mutation or is a variant associated with a disease.

6. The method of claim 1, wherein the endonuclease is a Cas endonuclease selected from the group consisting of Cas12a (from bacterial species Francisella novicida, Acidaminococcus or Lachnospiraceae), SaCas9 (from bacterial species Staphylococcus aureus), CjCas9 (from bacterial species Campylobacter jejuni), SpCas9 (from bacterial species Streptococcus pyogenes), and NmCas9 (from bacterial species Neisseria meningitidis), wherein the guide nucleic acid is RNA, and the target nucleic acid is a DNA or an RNA.

7. The method of claim 1, wherein amplifying is by using a polymerase having an optimal operating temperature substantially similar to an optimal operating temperature of the endonuclease.

8. The method of claim 1, wherein the method has between about 80% and 100% sensitivity of detecting target nucleic acids having a frequency of between about 0.1% and 5% in the sample.

9. A method of detecting a target nucleic acid in a sample, comprising:
amplifying the target nucleic acid in an amplification reaction comprising:
a) the target nucleic acid;
b) a non-target nucleic acid comprising protospaccr adjacent motif (PAM);
c) a guide nucleic acid comprising protospacer target sequence that forms a guide non-target hybrid with the non-target nucleic acid;
d) an endonuclease having an affinity for the guide/non-target hybrid.
e) a nucleic acid probe substantially specific to the target nucleic acid; and
f) a polymerase.

10. The method of claim 9, wherein the nucleic acid probe comprises a 3′ blocker, a detectable label comprising a fluorophore, a quencher, and an abasic nucleotide analogue between the fluorophore and the quencher, and
wherein the fluorophore and the quencher are separated by a length of between 2 and 10 nucleic acids.

11. The method of claim 9, wherein amplifying is by isothermal amplification for up to about 120 min, wherein the target nucleic acid in the sample is at a frequency between about 0.001% and about 15% of the nucleic acids in the sample, and wherein the frequency of the target nucleic acid is increased between 5(X) and 10,000 fold.

12. The method of claim 9, wherein the probe comprises a nucleic acid sequence substantially specific to nucleic acid sequence of the target nucleic acid and/or of the non-target nucleic acid.

13. The method of claim 9, wherein the abasic nucleotide analogue comprises tetrahydrofuran residue, THF, the fluorophore emits light at a wavelength between about 480 nm and 700 nm, and the quencher absorbs light at a w avelength betw een about 480 nm and 700 nm.

14. The method of claim 9, further comprising an exonuclease, and, optionally, wherein the exonuclease is an exonuclease III enzyme.

15. The method of claim 9, wherein the target nucleic acid comprises a mutation or is a variant associated with a disease, and wherein the target nucleic acid does not comprise PAM.

16. The method of claim 9, wherein amplifying the target nucleic acid comprises polymerase chain reaction (PCR), loop-mediated isothermal amplification (LAMP), recombinase polymerase amplification (RPA), or any combination thereof.

17. The method of claim 9, wherein the endonuclease is a Cas endonuclease selected from the group consisting of Cas12a (from bacterial species Francisella novicida, Acidaminococcus or Lachnospiraceae). SaCas9 (from bacterial species Staphylococcus aureus), CjCas9 (from bacterial species Campylobacter jejuni), SpCas9 (from bacterial species Streptococcus pyogenes), and NmCas9(from bacterial species Neisseria meningitidis), wherein the guide nucleic acid is RNA, and the target nucleic acid is a DNA or an RNA.

18. The method of claim 9, wherein the method has between about 80% and 100% sensitivity of real-time detecting of low frequency target nucleic acids having frequency of between about 0.1% and 5%.

19. The method of claim 1. wherein the method comprises amplifying two or more target nucleic acids in the same amplification reaction, and wherein the two or more target nucleic acids comprise mutant alleles. 20. A point-of-care assay comprising real-time detection of target nucleic acids in a sample according to the method of claim 9.
